(19) United States
(12) Patent Application Publication
(10) Pub. No.: US 2023/0338580 A1
(43) Pub. Date: Oct. 26, 2023

(54) PLASMID VECTORS AND NANOPARTICLES FOR TREATING OCULAR DISORDERS

(71) Applicant: CASE WESTERN RESERVE UNIVERSITY, Cleveland, OH (US)

(72) Inventors: Zheng-Rong LU, Cleveland, OH (US); Wenyu SUN, Cleveland, OH (US); Da SUN, Cleveland, OH (US)

(21) Appl. No.: 18/041,613

(22) PCT Filed: Aug. 16, 2021
(86) PCT No.: PCT/US21/46137

§ 371 Date: Feb. 14, 2023

Related U.S. Application Data
(60) Provisional application No. 63/065,860, filed on Aug. 14, 2020.

(51) Int. Cl. A61K 48/00; C12N 15/85; A61P 27/02; A61K 47/18; A61K 9/127

(52) U.S. Cl. CPC A61K 48/0058; A61K 48/0041; C12N 15/85; A61P 27/02; A61K 47/18; A61K 9/1272; A61K 38/1709

(57) ABSTRACT

A plasmid vector comprising one or more heterologous nucleic acids or genes encoding a functional therapeutic protein configured to treat a retinal or ocular includes a human GRK1 promoter and human S/MAR enhancer to express the heterologous gene in both rod and cone photoreceptors.

### RELATED APPLICATION

This application claims priority from U.S. Provisional Application No. 63/065,860, filed Aug. 14, 2020, the subject matter of which is incorporated herein by reference in its entirety.

### BACKGROUND

Gene replacement therapy, that delivers a healthy copy of a mutated gene into targeted cells and expresses the encoded functional protein to restore its normal function, has shown promise for effective treatment of Stargardt disease and other genetic ocular diseases. The first FDA approved gene therapy is adeno-associated virus (AAV) expressing hRPE65 for treating Leber's congenital amaurosis type 2 (LCA2). The recent success has re-energized the enthusiasm in developing gene therapy to treat previously untreatable genetic disorders. Numerous gene therapies have been developed and some are now in various phases of clinical trials. Most of the gene therapies under clinical development are based on AAVs. However, the broad application of AAV-based gene therapy is limited by its cargo capacity. This greatly restricted the application of viral gene therapies to treat ocular genetic diseases caused by mutations in large genes, such as Stargardt disease (STGD) and Usher Syndrome. For viral systems, strategies such as dual-AAV, multi-AAV vectors, and lentiviral vectors have been tested to overcome the limitations. Clinical application of these therapies is hindered by various limitations, including poor expression of whole functional proteins and immunogenicity. Non-viral gene delivery systems do not have limitations in gene packaging capacity and have also developed for treating various retinal genetic diseases covering wide range of gene sizes.

A challenge for gene therapy in retinal genetic diseases is to maintain prolonged stable protein expression to retain normal visual functions. Clinically, one subretinal administration of AAV therapy could last for years in LCA2 patients, but the clinical findings indicated declining therapeutic effect over time. Repetitive administrations may be needed to sustain the rescuing effect in retinal structure and function. Unfortunately, immune response development after the initial viral gene therapy renders the following repeated injection of the viral vector ineffective. Non-viral gene therapy exhibits low immunogenicity and can be repeatedly administered for prolonged therapeutic efficacy. However, non-viral gene delivery systems may suffer from low efficiency.

### SUMMARY

Embodiments described herein relate to a plasmid vector for use in expressing a functional therapeutic protein configured to treat a retinal or ocular disorder. The retinal or ocular disorder can be an inheritable retinal disorder caused by a mutation of a gene encoding a retinal or ocular protein. In some embodiments, the inheritable retinal disorder is selected from Stargardt Disease, Leber's congenital amaurosis (LCA), pseudoxanthoma elasticum, rod cone dystrophy, exudative vitreoretinopathy, Joubert Syndrome, CSNB-1C, retinitis pigmentosa, stickler syndrome, microcephaly, choriorretinopathy, CSNB 2, Usher syndrome, Wagner syndrome, or age-related macular degeneration.

The plasmid vector includes one or more heterologous nucleic acids or genes encoding a functional therapeutic protein configured to treat a retinal or ocular disorder, a human GRK1 promoter, and a human S/MAR enhancer to express the heterologous gene in both rod and cone photoreceptors.

In some embodiments, the human GRK1 promoter is upstream of the heterologous gene and the S/MAR enhancer is downstream of the heterologous gene.

In some embodiment, the plasmid vector can further include a human beta-globin polyadenylation (polyA) signal sequence. The human beta-globin polyA signal sequence can be downstream of the heterologous gene and upstream of the S/MAR enhancer.

In some embodiment, the heterologous gene is selected from Retinal pigment epithelium-specific 65 kDa protein (RPE65), vascular endothelial growth factor (VEGF) inhibitor or soluble VEGF receptor 1 (sFif1), (Rab escort protein-1) REP1, L-opsin, rhodopsin (Rho), phosphodiesterase 6(3 (PDE6I3), ATP-binding cassette, sub-family A, member 4 (ABCA4), lecithin retinol acyltransferase (LRAT), Retinal degeneration, slow/Peripherin (RDS/Peripherin), Tyrosine-protein kinase Mer (MERTK), Inosine-5 prime-monophosphate dehydrogenase, type I (IMPDHI), guanylate cyclase 2D (GUCY2D), aryl-hydrocarbon interacting protein-like 1 (AIPL 1), retinitis pigmentosa GTPase regulator interacting protein 1 (RPGRIPI), guanine nucleotide binding protein, alpha transducing activity polypeptide 2 (GNAT2), cyclic nucleotide gated channel beta 3 (CNGB3), retinoschisin 1 (Rs1), ocular albinism type 1 (OA1), oculocutaneous albinism type 1 (OCA1), tyrosinase, P21 WAF-1/Cip1, platelet-derived growth factor (PDGF), Endostatin, Angiostatin, arylsulfatase B, 13-glucuronidase, usherin 2A (USH2A), centrosomal protein 290 (CEP290), regulating synaptic membrane exocytosis 1 (RIMS1), LDL receptor related protein 5 (LRP5), Coiled-coil and C2 domain containing 2A (CC2D2A), transient receptor potential cation channel subfamily M member 1 (TRPM1), intraflagellar transport 172 (IFT-172), collagen type 1 alpha 1 chain (COL11A1), tubulin gamma complex associated protein 6 (TUBGCP6), KIAA1549, calcium voltage-gated channel subunit alpha 1 F (CACNA1F), myosin VIIA (MYO7A), versican (VCAN), or hemicentin 1 (HMCN1).

In some embodiments, the heterologous gene is ABCA4 and the retinal disorder is Stargardt Disease or age-related macular degeneration.

In some embodiments, the plasmid has the nucleic acid sequence of SEQ ID NO: 1.

Other embodiments described herein relate to a self-assembled nanoparticle comprising a plurality of pH sensitive multifunctional cationic lipids complexed with one or more plasmid vector(s) described herein.

In some embodiments, the pH sensitive multifunctional cationic lipids can include pH sensitive multifunctional amino lipids.

In some embodiments, the self-assembled nanoparticles can have an amine to phosphate (N/P) ratio of about 4 to about 12, preferably, about 6 to about 10.

In some embodiments, the pH sensitive multifunctional cationic lipids can further include at least one targeting group that targets and/or binds to a retinal or visual protein. The at least one targeting group can include a retinoid or retinoid derivative that targets and/or binds to an interphotoreceptor retinoid binding protein. For example, the at least one targeting group includes all-trans-retinylamine or (1R)-3-amino-1-[3-(cyclohexylmethoxy)phenyl]propan-1-ol.

In some embodiments, the pH sensitive multifunctional cationic lipids include a cysteine residue and the at least one targeting group is covalently attached to a thiol group of the cysteine residue by a linker. The linker can include, for example, a polyamino acid group, a polyalkylene group, or a polyethylene glycol group. Optionally, the linker can include an acid labile bond.

In some embodiments, the self-assembled nanoparticles are PEGylated.

In some embodiments, the pH sensitive multifunctional cationic lipids can include (1-aminoethyl)iminobis[N-(oleoylcysteinyl-1-amino-ethyl)propionamide) (ECO) or an analogue or derivative thereof.

Ins some embodiments, ECO as well as analogues or derivatives thereof can include a compound that has formula (I):

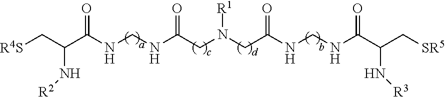

wherein R¹ is an alkylamino group or a group containing at least one aromatic group;

R² and R³ are independently an aliphatic group or a hydrophobic group;

R⁴ and R⁵ are independently H, a substituted or unsubstituted alkyl group, an alkenyl group, an acyl group, or an aromatic group, or includes a polymer, a targeting group, or a detectable moiety;

a, b, c, and d are independently an integer from 1 to 10; and pharmaceutically acceptable salts thereof.

In some embodiments, R¹ comprises at least one of:

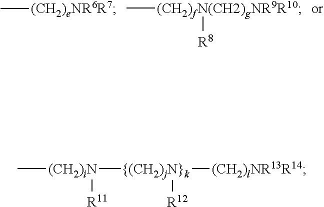

where R⁶, R⁷, R⁸, R⁹, R¹⁰, R¹¹, R¹², R¹³, and R¹⁴ are independently hydrogen, an alkyl group, a hydrophobic group, or a nitrogen containing substituent; and

e, f, g, i, j, k, and 1, are an integer from 1 to 10.

In other embodiments, R² and R³ are independently a hydrophobic group derived from oleic acid or linoleic acid.

In some embodiments, R² and R³ are the same.

In some embodiments, at least one of R⁴ and R⁵ includes a targeting group that targets and/or binds to a retinal or visual protein. At least one of R⁴ or R⁵ can include a retinoid or retinoid derivative that targets and/or binds to an interphotoreceptor retinoid binding protein. For example, at least one of R⁴ or R⁵ includes all-trans-retinylamine or (1R)-3-amino-1-[3-(cyclohexylmethoxy)phenyl]propan-1-ol.

In some embodiments, a, b, c, and d are each 2.

In other embodiments, R¹ comprises at least one of CH₂CH₂NH₂, CH₂CH₂NHCH₂CH₂NHCH₂CH₂NH, or CH₂CH₂NHCH₂CH₂CH₂CH₂NHCH₂CH₂CH₂NH.

In some embodiments, the targeting group is covalently attached to a thiol group of a cysteine residue by a linker. The linker can include a polyamino acid group, a polyalkylene group, or a polyethylene glycol group and optionally an acid labile bond.

Still other embodiments, relate to a pharmaceutical composition comprising an aqueous solution of the self-assembled nanoparticles described herein and/or the plasmid vectors described herein.

In some embodiments, the pharmaceutical composition can include an amount of sucrose effective to enhance the stability under different or differing storage temperatures. The different or differing storage temperatures can range from about −20° C. to about 4° C. For example, the pharmaceutical composition can include about 5% to about 20% sucrose.

In some embodiments, pharmaceutical composition can have a substantially neutral pH. The substantially neutral pH is about 6.0 to about 8.0, about 6.2 to about 7.8, about 6.5 to about 7.5, about 6.8 to about 7.2, or about 7.

In some embodiments, the pharmaceutical composition is free of an excipient besides sucrose.

In other embodiments, the pharmaceutical composition includes a concentration of self-assembled nanoparticles of about 50 ng/μl to about 500 ng/μl, about 100 ng/μl to about 300 ng/μl, or about 150 ng/μl to about 250 ng/μl.

Other embodiments described herein relate to a method of inducing episomal expression of a heterologous gene in a subject in need thereof. The method can include administering to the subject the plasmid vectors, the self-assembled nanoparticles, or the pharmaceutical composition described.

Still other embodiments described herein relate to a method of treating a disorder in a subject. The method includes administering to the subject the plasmid vectors, the self-assembled nanoparticles, or the pharmaceutical composition described.

In some embodiments, the plasmid vectors, the self-assembled nanoparticles, or the pharmaceutical composition are administered repeatedly.

In other embodiments, the plasmid vectors, the self-assembled nanoparticles, or the pharmaceutical composition are administered in a single dose.

In some embodiments, the plasmid vectors, the self-assembled nanoparticles, or the pharmaceutical composition are administered locally. For example, the self-assembled nanoparticles, or the pharmaceutical composition can be administered intravitreally, subretinally, or suprachoroidally.

In some embodiments, the disorder treated by the method can be an inheritable retinal disorder caused by a mutation of a gene encoding a retinal or ocular protein. In some embodiments, the inheritable retinal disorder is selected from Stargardt Disease, Leber's congenital amaurosis (LCA), pseudoxanthoma elasticum, rod cone dystrophy, exudative vitreoretinopathy, Joubert Syndrome, CSNB-1C, retinitis pigmentosa, stickler syndrome, microcephaly, choriorretinopathy, CSNB 2, Usher syndrome, Wagner syndrome, or age-related macular degeneration.

In some embodiments, the concentration of the self-assembled nanoparticles in the pharmaceutical composition is about 50 ng/μl to about 500 ng/μl, about 100 ng/μl to about 300 ng/μl, or about 150 ng/μl to about 250 ng/μl.

In other embodiments, the volume of the pharmaceutical composition administered to the subject is about 0.1 μL to about 200 μL, about 0.2 μL to about 150 μL, or about 0.5 μL to about 100 μL.

In some embodiments, the plasmid vectors, the self-assembled nanoparticles, or the pharmaceutical composition can be effective in treating the subject's visual function. The visual function can be assessed by microperimetry, dark-adapted perimetry, assessment of visual mobility, visual acuity, ERG, or reading assessment.

In some embodiments, the method results in the prevention of or a slowing of the progression of decline of the subject's visual function due to progression of the ocular disorder.

### BRIEF DESCRIPTION OF THE DRAWINGS

FIG. 1 is a diagram of pGRK1-ABCA4-SMAR plasmid. GRK1 promoter was inserted between MluI and AgeI restriction sites. S/MAR DNA was inserted between NotI and NheIrestriction sites. Globin polyA DNA was inserted by using NEB HiFi assembly cloning kit between NotI and SpeI restriction sites.

FIGS. 2(A-B) illustrate agarose gel electrophoresis of multiple restriction enzyme digestion to verify correct-sized DNA fragments in target plasmid. (A) agarose gel (1%) to separate 4 DNA fragments after digestion with the promoter GRK1 (400 bp), GFP (700 bp), ABCA4 fragment (6.8 kb), polyA+SMAR (2.9 kb), and the vector backbone (˜2.5 kb). (B) a longer run of the same agarose gel to separate backbone and pA+SMAR fragments for a clearer view. (Four restriction enzymes were used: MluI, AgeI, NotI and NheI for multiple enzyme digestion.)

FIG. 3 is a schematic of the preparation of ECO/pGRK1-ABCA4-S/MAR nanoparticles. ECO self-assembles with pGRK1-ABCA4-S/MAR in aqueous solution at predetermined pDNA concentration and amine/phosphate (N/P) ratio to form stable nanoparticles.

FIGS. 4(A-C) ECO/pGRK1-ABCA4-S/MAR nanoparticle (N/P=8) formulation. DLS of size distribution (A), zeta potential distribution (B) and plasmid encapsulation (C) by agarose gel electrophoresis.

FIGS. 5(A-B) ABCA4 expression in abca4^{-/-}mice after subretinal administration of ECO/pGRK1-ABCA4-S/MAR nanoparticles (200 ng/μL, 1 μL) for 7 days. Confocal microscopic images of ABCA4 expressions at different locations in the retina (A) and whole retina (B). Immunostaining with polyclonal ABCA4 antibody. ABCA4 expression was demonstrated in white color. PBS injected eye samples were used as controls.

FIG. 6 illustrates ABCA4 expression in abca4^{-/-}mice after subretinal administration of ECO/pGRK1-ABCA4-S/MAR nanoparticles (400 ng/μL, 0.5 μL) for 8 days. qRT-PCR analysis of ABCA4 mRNA expression. ABCA4 mRNA levels were normalized to the contralateral PBS control and were demonstrated individually.

FIG. 7 illustrates ABCA4 expression in abca4^{-/-}mice after subretinal administration of ECO/pGRK1-ABCA4-S/MAR (10% sucrose) nanoparticles (200 ng/μL, 1 μL) for 7 days. qRT-PCR analysis of ABCA4 mRNA expression. ABCA4 mRNA levels were normalized to the contralateral PBS control and were demonstrated individually.

FIG. 8 is a schematic of the preparation of PEGylated PEG-ECO/pGRK1-ABCA4-S/MAR nanoparticles. PEG-MAL ligand (2.5 mol-%) is first mixed and reacted with ECO molecules in aqueous solution for 30 min. The PEGylated nanoparticles then formed by self-assembly when pGRK1-ABCA4-S/MAR was added to the solution at predetermined pDNA concentration and amine/phosphate (N/P) ratio to form stable nanoparticles.

FIGS. 9(A-D) illustrate stability of ECO/pGRK1-ABCA4-S/MAR and PEG-ECO/pGRK1-ABCA4-S/MAR nanoparticles with or without 10% sucrose under different storage temperatures. DLS of size distributions of ECO/pGRK1-ABCA4-S/MAR nanoparticles (A), PEG-ECO/pGRK1-ABCA4-S/MAR nanoparticles (B), ECO/pGRK1-ABCA4-S/MAR nanoparticles (10% sucrose) (C) and PEG-ECO/pGRK1-ABCA4-S/MAR (10% sucrose) nanoparticles (D) stored at 4° C. and −20° C., which were tested at day 0, day 7 and 1 month.

FIG. 10 illustrates agarose gel electrophoresis of ECO/pGRK1-ABCA4-S/MAR and PEG-ECO/pGRK1-ABCA4-S/MAR nanoparticles with or without 10% sucrose under 4 or −20° C. at day 0, day 7 and 1 month.

FIG. 11 illustrates ABCA4 expression in abca4^{-/-}mice after subretinal administration of ECO/pGRK1-ABCA4-S/MAR, PEGylated (2.5% PEG) PEG-ECO/pGRK1-ABCA4-S/MAR and ECO/pGRK1-ABCA4-S/MAR (5% sucrose) nanoparticles with qRT-PCR analysis of ABCA4 mRNA expression. (The mRNA levels of NP1 was normalized to NP2. The mRNA levels of NP3 was normalized to NP1.)

FIG. 12 illustrates ABCA4 expression in abca4^{-/-}mice after subretinal administration of PEGylated PEG-ECO/pGRK1-ABCA4-S/MAR nanoparticles at the same dose but different volumes. qRT-PCR analysis of ABCA4 mRNA expression with ABCA4 mRNA levels of 1 μL injection volume normalized to 0.5 μL injection volume.

FIG. 13 illustrates ABCA4 mRNA expression 7 days after subretinal injection of PEGylated (2.5%) PEG-ECO/pGRK1-ABCA4-S/MAR nanoparticles. The mRNA levels were normalized to 50 ng dose group.

FIG. 14 illustrates ABCA4 mRNA expressions of abca4^{-/-}mice with different ages 7 days after subretinal injection of PEGylated PEG-ECO/pGRK1-ABCA4-S/MAR (2.5% PEG) nanoparticles.

FIG. 15 illustrates ABCA4 expression in abca4^{-/-}mice after subretinal administrations of PEGylated (2.5% PEG) PEG-ECO/pGRK1-ABCA4-S/MAR nanoparticles stored under −20° C. for 1 week, 2 weeks and 1 month demonstrated by qRT-PCR analysis of ABCA4 mRNA expression (Fresh prepared nanoparticles were used as controls).

FIG. 16 illustrates ABCA4 expression in abca4^{-/-}mice after subretinal administration of PEGylated (2.5% PEG) PEG-ECO/pGRK1-ABCA4-S/MAR nanoparticles with qRT-PCR analysis of ABCA4 mRNA expression. (The mRNA levels were normalized to untreated control mice).

FIG. 17 illustrates ABCA4 expression in abca4^{-/-}mice after intravitreal administration of PEGylated (2.5% PEG) PEG-ECO/pGRK1-ABCA4-S/MAR nanoparticles with qRT-PCR analysis of ABCA4 mRNA expression. (The mRNA levels were normalized to untreated control mice).

FIG. 18 illustrates comparison of ABCA4 expression in abca4^{-/-}mice after subretinal and intravitreal administrations of PEGylated (2.5% PEG) PEG-ECO/pGRK1-ABCA4-S/MAR nanoparticles with qRT-PCR analysis of ABCA4 mRNA expression. (The mRNA levels were normalized to untreated control mice).

FIG. 19 illustrates efficacy of PEG-ECO/pGRK1-ABCA4-S/MAR nanoparticles for preventing A2E accumulation in abca4^{-/-}mice. Quantitative A2E levels of PEG-ECO/pGRK1-ABCA4-S/MAR nanoparticles treated abca4^{-/-}mice relative to control mice 8 months after subretinal injections.

FIG. 20 illustrates efficacy of PEG-ECO/pGRK1-ABCA4-S/MAR nanoparticles for preventing A2E accumulation in abca4^{-/-}mice. HPLC chromatograms of A2E from control and nanoparticles treated abca4^{-/-}mice 8 months after subretinal injections.

FIG. 21 illustrates ABCA4 expression in abca4^{-/-}mice 8 months after subretinal administration of PEG-ECO/pGRK1-ABCA4-S/MAR nanoparticles (100 ng). ABCA4 expression was demonstrated in green color. RPE65 was labeled with red.

FIG. 22 illustrates efficacy of PEG-ECO/pGRK1-ABCA4-S/MAR nanoparticles for preventing A2E accumulation in abca4^{-/-}mice. Quantitative A2E levels of PEG-ECO/pGRK1-ABCA4-S/MAR nanoparticles treated abca4^{-/-}mice relative to control mice 1 year after subretinal injections.

FIG. 23 illustrates efficacy of PEG-ECO/pGRK1-ABCA4-S/MAR nanoparticles in abca4^{-/-}mice. ABCA4 mRNA expression 1 year after subretinal treatments.

FIG. 24 illustrates efficacy of PEG-ECO/pGRK1-ABCA4-S/MAR nanoparticles for preventing A2E accumulation in abca4^{-/-}mice after multi-treatments. Quantitative A2E levels of PEG-ECO/pGRK1-ABCA4-S/MAR nanoparticles treated abca4^{-/-}mice relative to control mice 8 months after the first subretinal treatment. (*p<0.05, **p<0.005, ***p<0.0005, ****p<0.0001)

FIG. 25 illustrates efficacy of PEG-ECO/pGRK1-ABCA4-S/MAR nanoparticles for preventing A2E accumulation in abca4^{-/-}mice after multi-treatments. HPLC chromatograms of A2E from control and nanoparticles treated Abca4^{-/-}mice 8 months after the first subretinal injection.

FIG. 26 illustrates efficacy of PEG-ECO/pGRK1-ABCA4-S/MAR nanoparticles in abca4^{-/-}mice after multi-treatments. ABCA4 mRNA expression 8 year after the first subretinal treatment.

FIG. 27 illustrates safety of PEG-ECO/pGRK1-ABCA4-S/MAR nanoparticles. In vivo safety of abca4^{-/-}mice receiving gene therapies of PEG-ECO/pGRK1-ABCA4-S/MAR nanoparticles demonstrated by scanning laser ophthalmoscopy (SLO) images. Images were taken through fluorescent (F) and infrared (IR) channels 7 months after a single dose subretinal injection of 100 ng. Controls are the untreated mice. No adverse effect was observed for the treated mice.

FIG. 28 illustrates safety of PEG-ECO/pGRK1-ABCA4-S/MAR nanoparticles after multi-treatments. In vivo safety of abca4^{-/-}mice receiving gene therapies of PEG-ECO/pGRK1-ABCA4-S/MAR nanoparticles demonstrated by scanning laser ophthalmoscopy (SLO) images. Images were taken through fluorescent (F) and infrared (IR) channels 8 months after the first dose of subretinal injection of 100 ng. Mice were injected every 3 months, with total 2 injections.

FIG. 29 illustrates safety of PEG-ECO/pDNA nanoparticles. Eye condition and GFP expression in the eyes of BALB/c mice treated with ACU-PEG-HZ-ECO/pCMV-GFP nanoparticles with 5% sucrose. AAV2-CMV-GFP was used as a positive control. Scanning laser ophthalmoscope (SLO) images of eye conditions and GFP expression 1, 2 and 3 months after treatments. White dots (GFP), large white area (potential inflammation).

FIG. 30 illustrates size distributions of the nanoparticles of ECO with pCMV-ABCA4, pCMV-ABCA4-SV40, pRHO-ABCA4, and pRHO-ABCA4-SV40 in the presence of sucrose and sorbitol as measured by dynamic light scattering.

FIG. 31 illustrates zeta potential of the nanoparticle formulations of ECO with pCMV-ABCA4, pCMV-ABCA4-SV40, pRHO-ABCA4, and pRHO-ABCA4-SV40 in existence with sucrose and sorbitol as stabilizers.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present invention.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like. “Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

The term “alkenyl group” is defined herein as a C₂-C₂₀ alkyl group possessing at least one C═C double bond.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “acyl” group as used herein is represented by the formula C(O)R, where R is an organic group such as, for example, an alkyl or aromatic group as defined herein.

The term “alkylene group” as used herein is a group having two or more CH₂ groups linked to one another. The alkylene group can be represented by the formula (CH₂)ₐ, where a is an integer of from 2 to 25.

The term “aromatic group” as used herein is any group containing an aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The phrase “nitrogen containing substituent” is defined herein as any amino group. The term “amino group” is defined herein as a primary, secondary, or tertiary amino group. In the alternative, the nitrogen containing substituent can be a quaternary ammonium group. The nitrogen containing substituent can be an aromatic or cycloaliphatic group, where the nitrogen atom is either part of the ring or directly or indirectly attached by one or more atoms (i.e., pendant) to the ring. The nitrogen containing substituent can be an alkylamino group having the formula RNH₂, where R is a branched or straight alkyl group, and the amino group can be substituted or unsubstituted.

The term “nucleic acids,” “nucleic acid molecules,” “nucleotides,” “nucleotide(s) sequence,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region, or comprise two or more coding regions.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

The term “heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “heterologous gene” or “heterologous nucleic acid” are used interchangeably and refers to a gene or nucleic acid that does not naturally occur as part of a viral genome. For instance, a heterologous gene can be a mammalian gene, e.g., a therapeutic gene, e.g., a mammalian gene that encodes a therapeutic protein. In some embodiments, a heterologous gene encodes a protein or portion thereof that is defective or absent in the target cell and/or subject. In some embodiments, the heterologous gene contains one or more exons encoding a protein that is defective or absent in the target cell and/or subject. For example, in some embodiments, the heterologous gene includes one or more trans-splicing molecules, e.g., as described in WO 2017/087900, which is incorporated herein by reference in its entirety. In some embodiments, a heterologous gene includes a therapeutic nucleic acid, such as a therapeutic RNA (e.g., microRNA).

The term “promoter” refers to a sequence that regulates transcription of a heterologous gene operably linked to the promoter. Promoters provide the sequence sufficient to direct transcription and/or recognition sites for RNA polymerase and other transcription factors required for efficient transcription and can direct cell-specific expression. In addition to the sequence sufficient to direct transcription, a promoter sequence can also include sequences of other regulatory elements that are involved in modulating transcription (e.g., enhancers, kozak sequences, and introns).

The term “target cell” refers to any cell that expresses a target gene and which the vector infects or is intended to infect. Vectors can infect target cells that reside in a subject (in situ) or target cells in culture. In some embodiments, target cells of the invention are post-mitotic cells. Target cells include both vertebrate and invertebrate animal cells (and cell lines of animal origin). Representative examples of vertebrate cells include mammalian cells, such as humans, rodents (e.g., rats and mice), and ungulates (e.g., cows, goats, sheep and swine). Target cells include ocular cells, such as retinal cells. Alternatively, target cells can be stem cells (e.g., pluripotent cells (i.e., a cell whose descendants can differentiate into several restricted cell types, such as hematopoietic stem cells or other stem cells) or totipotent cells (i.e., a cell whose descendants can become any cell type in an organism, e.g., embryonic stem cells, and somatic stem cells e.g., hematopoietic cells)). In yet other embodiments, target cells include oocytes, eggs, cells of an embryo, zygotes, sperm cells, and somatic (non-stem) mature cells from a variety of organs or tissues, such as hepatocytes, neural cells, muscle cells and blood cells (e.g., lymphocytes).

The term “host cell” as used herein refers to, for example microorganisms, yeast cells, insect cells, and mammalian cells, that can be, or have been, used as recipients of ssDNA or vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation. In some embodiments, the host cell can be an in vitro host cell.

The terms “disorder associated with a mutation” or “mutation associated with a disorder” refer to a correlation between a disorder and a mutation. In some embodiments, a disorder associated with a mutation is known or suspected to be wholly or partially, or directly or indirectly, caused by the mutation. For example, a subject having the mutation may be at risk of developing the disorder, and the risk may additionally depend on other factors, such as other (e.g., independent) mutations (e.g., in the same or a different gene), or environmental factors.

The term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, aves, etc.), which is to be the recipient of a particular treatment. Typically, the terms “patient” and “subject” are used interchangeably herein in reference to a human subject.

The term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

All percentages and ratios used herein, unless otherwise indicated, are by weight.

Embodiments described herein relate to plasmid vectors for use in expressing a functional therapeutic protein configured to treat a retinal or ocular disorder, self-assembled nanoparticles that include a plurality of pH sensitive multifunctional cationic lipids complexed with one or more plasmid vector(s) described herein, a pharmaceutical composition comprising an aqueous solution of the self-assembled nanoparticles described herein and/or the plasmid vectors described herein, and methods of inducing episomal expression of a heterologous gene or treating a retinal or ocular disorder in a subject in need thereof.

In some embodiments, the plasmid vector can provide long-term transcription or expression of a heterologous gene of vector. In some embodiments, the persistence of the plasmid vector is from 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, the plasmid vector described herein persists for one week to four weeks, from one month to four months, from four months to one year, from one year to five years, from five years to twenty years, or from twenty years to fifty years (e.g., at least one week, at least two weeks, at least one month, at least four months, at least one year, at least two years, at least five years, at least ten years, at least twenty years, at least thirty years, at least forty years, or at least fifty years).

The plasmid vector can be a circular plasmid vector. The circular plasmid vector may be monomeric, dimeric, trimeric, tetrameric, pentameric, hexameric, etc. Preferably, the circular plasmid vector is monomeric or dimeric. In other embodiments, the circular plasmid vector is a monomeric, supercoiled circular molecule. In some embodiments, the plasmid vector can be nicked. In some embodiments, the plasmid vector can be open circular. In some embodiments, the plasmid vector can be double-stranded circular.

The plasmid vectors described herein can be used to insert or express a heterologous gene into a target cell. As disclosed herein, a broad range of heterologous genes may be delivered to target cells by way of the present vectors. In some embodiments, the heterologous gene is configured to transfect a target cell having a mutation associated with a disease which can be treated by expression of the heterologous gene, e.g., a gene encoding a therapeutic protein, e.g., a protein that is defective or absent in the target cell and/or subject.

In some embodiments, the heterologous gene or nucleic acid may encode for a viable therapeutic protein so as to replace the defective protein which is naturally expressed in the targeted tissue. Typically, defective genes that may be replaced include, but are not limited to, genes that are responsible for retinal degenerative diseases such as retinitis pigmentosa (RP), Leber congenital amaurosis (LCA), recessive RP, Dominant retinitis pigmentosa, X-linked retinitis pigmentosa, Incomplete X-linked retinitis pigmentosa, dominant, Dominant Leber congenital amaurosis, Recessive ataxia, posterior column with retinitis pigmentosa, Recessive retinitis pigmentosa with para-arteriolar preservation of the RPE, Retinitis pigmentosa RP12, Usher syndrome, Dominant retinitis pigmentosa with sensorineural deafness, Recessive retinitis punctata albescens, Recessive Alstrom syndrome, Recessive Bardet-Biedl syndrome, Dominant spinocerebellar ataxia w/macular dystrophy or retinal degeneration, Recessive abetalipoproteinemia, Recessive retinitis pigmentosa with macular degeneration, Recessive Refsum disease, adult form, Recessive Refsum disease, infantile form, Recessive enhanced S-cone syndrome, Retinitis pigmentosa with mental retardation, Retinitis pigmentosa with myopathy, Recessive Newfoundland rod-cone dystrophy, Retinitis pigmentosa sinpigmento, Sector retinitis pigmentosa, Regional retinitis pigmentosa, Senior-Loken syndrome, Joubert syndrome, Stargardt disease, juvenile, Stargardt disease, late onset, Dominant macular dystrophy, Stargardt type, Dominant Stargardt-like macular dystrophy, Recessive macular dystrophy, Recessive fundus flavimaculatus, Recessive cone-rod dystrophy, X-linked progressive cone-rod dystrophy, Dominant cone-rod dystrophy, Cone-rod dystrophy; de Grouchy syndrome, Dominant cone dystrophy, X-linked cone dystrophy, Recessive cone dystrophy, Recessive cone dystrophy with supernormal rod electroretinogram, X-linked atrophic macular dystrophy, X-linked retinoschisis, Dominant macular dystrophy, Dominant radial, macular drusen, Dominant macular dystrophy, bull's-eye, Dominant macular dystrophy, butterfly-shaped, Dominant adult vitelliform macular dystrophy, Dominant macular dystrophy, North Carolina type, Dominant retinal-cone dystrophy 1, Dominant macular dystrophy, cystoid, Dominant macular dystrophy, atypical vitelliform, Foveomacular atrophy, Dominant macular dystrophy, Best type, Dominant macular dystrophy, North Carolina-like with progressive, Recessive macular dystrophy, juvenile with hypotrichosis, Recessive foveal hypoplasia and anterior segment dysgenesis, Recessive delayed cone adaptation, Macular dystrophy in blue cone monochromacy, Macular pattern dystrophy with type II diabetes and deafness, Flecked Retina of Kandori, Pattern Dystrophy, Dominant Stickler syndrome, Dominant Marshall syndrome, Dominant vitreoretinal degeneration, Dominant familial exudative vitreoretinopathy, Dominant vitreoretinochoroidopathy; Dominant neovascular inflammatory vitreoretinopathy, Goldmann-Favre syndrome, Recessive achromatopsia, Dominant tritanopia, Recessive rod monochromacy, Congenital red-green deficiency, Deuteranopia, Protanopia, Deuteranomaly, Protanomaly, Recessive Oguchi disease, Dominant macular dystrophy, late onset, Recessive gyrate atrophy, Dominant atrophia greata, Dominant central areolar choroidal dystrophy, X-linked choroideremia, Choroidal atrophy, Central areolar, Central, Peripapillary, Dominant progressive bifocal chorioretinal atrophy, Progresive bifocal Choroioretinal atrophy, Dominant Doyne honeycomb retinal degeneration (Malattia Leventinese), Amelogenesis imperfecta, Recessive Bietti crystalline corneoretinal dystrophy, Dominant hereditary vascular retinopathy with Raynaud phenomenon and migraine, Dominant Wagner disease and erosive vitreoretinopathy, Recessive microphthalmos and retinal disease syndrome; Recessive nanophthalmos, Recessive retardation, spasticity and retinal degeneration, Recessive Bothnia dystrophy, Recessive pseudoxanthoma elasticum, Dominant pseudoxanthoma elasticum; Recessive Batten disease (ceroid-lipofuscinosis), juvenile, Dominant Alagille syndrome, McKusick-Kaufman syndrome, hypoprebetalipoproteinemia, acanthocytosis, palladial degeneration; Recessive Hallervorden-Spatz syndrome; Dominant Sorsby's fundus dystrophy, Oregon eye disease, Kearns-Sayre syndrome, Retinitis pigmentosa with developmental and neurological abnormalities, Basseb Korenzweig Syndrome, Hurler disease, Sanfilippo disease, Scieie disease, Melanoma associated retinopathy, Sheen retinal dystrophy, Duchenne macular dystrophy, Becker macular dystrophy, and Birdshot Retinochoroidopathy.

Examples of heterologous genes that encode for a viable therapeutic protein so as to replace the defective protein associated with the retinal or ocular disorder are selected from Retinal pigment epithelium-specific 65 kDa protein (RPE 65), vascular endothelial growth factor (VEGF) inhibitor or soluble VEGF receptor 1 (sFif1), (Rab escort protein-1) REP1, L-opsin, rhodopsin (Rho), phosphodiesterase 6(3 (PDE6I3), ATP-binding cassette, sub-family A, member 4 (ABCA4), lecithin retinol acyltransferase (LRAT), Retinal degeneration, slow/Peripherin (RDS/Peripherin), Tyrosine-protein kinase Mer (MERTK), Inosine-5 prime-monophosphate dehydrogenase, type I (IMPDHI), guanylate cyclase 2D (GUCY2D), aryl-hydrocarbon interacting protein-like 1 (AIPL 1), retinitis pigmentosa GTPase regulator interacting protein 1 (RPGRIPI), guanine nucleotide binding protein, alpha transducing activity polypeptide 2 (GNAT2), cyclic nucleotide gated channel beta 3 (CNGB3), retinoschisin 1 (Rs1), ocular albinism type 1 (OA1), oculocutaneous albinism type 1 (OCA1), tyrosinase, P21 WAF-1/Cip1, platelet-derived growth factor (PDGF), Endostatin, Angiostatin, arylsulfatase B, B-glucuronidase, usherin 2A (USH2A), centrosomal protein 290 (CEP290), regulating synaptic membrane exocytosis 1 (RIMS1), LDL receptor related protein 5 (LRP5), Coiled-coil and C2 domain containing 2A (CC2D2A), transient receptor potential cation channel subfamily M member 1 (TRPM1), intraflagellar transport 172 (IFT-172), collagen type 1 alpha 1 chain (COL11A1), tubulin gamma complex associated protein 6 (TUBGCP6), KIAA1549, calcium voltage-gated channel subunit alpha 1 F (CACNA1F), myosin VIIA (MYO7A), versican (VCAN), or hemicentin 1 (HMCN1).

Other examples of therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Various retina-derived neurotrophic factors have the potential to rescue degenerating photoreceptor cells, and may be expressed using a vector as described herein. Preferred biologically active agents may be selected from VEGF, Angiogenin, Angiopoietin-1, DeM, acidic or basic Fibroblast Growth Factors (aFGF and bFGF), FGF-2, Follistatin, Granulocyte Colony-Stimulating factor (G-CSF), Hepatocyte Growth Factor (HGF), Scatter Factor (SF), Leptin, Midkine, Placental Growth Factor (PGF), Platelet-Derived Endothelial Cell Growth Factor (PD-ECGF), Platelet-Derived Growth Factor-BB (PDGF-BB), Pleiotrophin (PTN), RdCVF (Rod-derived Cone Viability Factor), Progranulin, Proliferin, Transforming Growth Factor-alpha (TGF-alpha), PEDF, Transforming Growth Factor-beta (TGF-beta), Tumor Necrosis Factor-alpha (TNF-alpha), Vascular Endothelial Growth Factor (VEGF), Vascular Permeability Factor (VPF), CNTF, BDNF, GDNF, PEDF, NT3, BFGF, angiopoietin, ephrin, EPO, NGF, IGF, GMF, aFGF, NT5, Gax, a growth hormone, [alpha]-1-antitrypsin, calcitonin, leptin, an apolipoprotein, an enzyme for the biosynthesis of vitamins, hormones or neuromediators, chemokines, cytokines such as IL-1, IL-8, IL-10, IL-12, IL-13, a receptor thereof, an antibody blocking any one of said receptors, TIMP such as TIMP-1, TIMP-2, TIMP-3, TIMP-4, angioarrestin, endostatin such as endostatin XVIII and endostatin XV, ATF, angiostatin, a fusion protein of endostatin and angiostatin, the C-terminal hemopexin domain of matrix metalloproteinase-2, the kringle 5 domain of human plasminogen, a fusion protein of endostatin and the kringle 5 domain of human plasminogen, the placental ribonuclease inhibitor, the plasminogen activator inhibitor, the Platelet Factor-4 (PF4), a prolactin fragment, the Proliferin-Related Protein (PRP), the antiangiogenic antithrombin III, the Cartilage-Derived Inhibitor (CDI), a CD59 complement fragment, C3a and C5a inhibitors, complex attack membrane inhibitors, Factor H, ICAM, VCAM, caveolin, PKC zeta, junction proteins, JAMs, CD36, MERTK vasculostatin, vasostatin (calreticulin fragment), thrombospondin, fibronectin, in particular fibronectin fragment gro-beta, an heparinase, human chorionic gonadotropin (hCG), interferon alpha/beta/gamma, interferon inducible protein (IP-10), the monokine-induced by interferon-gamma (Mig), the interferon-alpha inducible protein 10 (IP10), a fusion protein of Mig and IP10, soluble Fms-Like Tyrosine kinase 1 (FLT-1) receptor, Kinase insert Domain Receptor (KDR), regulators of apoptosis such as Bc1-2, Bad, Bak, Bax, Bik, BcI-X short isoform and Gax, fragments or derivatives thereof and the like.

In other embodiments, the heterologous gene can encode a site-specific endonuclease that provides for site-specific knock-down of gene function, e.g., where the endonuclease knocks out an allele associated with a retinal disease. For example, where a dominant allele encodes a defective copy of a gene that, when wild-type, is a retinal structural protein and/or provides for normal retinal function, a site-specific endonuclease (such as TALEnucleases, meganucleases or Zinc finger nucleases) can be targeted to the defective allele and knock out the defective allele. In addition to knocking out a defective allele, a site-specific nuclease can also be used to stimulate homologous recombination with a donor DNA that encodes a functional copy of the protein encoded by the defective allele. Thus, e.g., the method of the invention can be used to deliver both a site-specific endonuclease that knocks out a defective allele, and can be used to deliver a functional copy of the defective allele, resulting in repair of the defective allele, thereby providing for production of a functional retinal protein (e.g., functional retinoschisin, functional RPE65, functional peripherin, etc.). See, e.g., Li et al. (2011) Nature 475:217. In some embodiments, the vector comprises a polynucleotide that encodes a site-specific endonuclease; and a polynucleotide that encodes a functional copy of a defective allele, where the functional copy encodes a functional retinal protein. Functional retinal proteins include, e.g., retinoschisin, RPE65, retinitis pigmentosa GTPase regulator (RGPR)-interacting protein-1, peripherin, peripherin-2, and the like. Site-specific endonucleases that are suitable for use include, e.g., zinc finger nucleases (ZFNs); and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Patent Publication No. 2011/0301073.

In other embodiments, the heterologous gene can encode an antibody, or a portion, fragment, or variant thereof. Antibodies include fragments that are capable of binding to an antigen, such as Fv, single-chain Fv (scFv), Fab, Fab′, di-scFv, sdAb (single domain antibody) and (Fab′)₂ (including a chemically linked F(ab′)₂). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. Antibodies also include chimeric antibodies and humanized antibodies. Furthermore, for all antibody constructs provided herein, variants having the sequences from other organisms are also contemplated. Thus, if a human version of an antibody is disclosed, one of skill in the art will appreciate how to transform the human sequence based antibody into a mouse, rat, cat, dog, horse, etc. sequence. Antibody fragments also include either orientation of single chain scFvs, tandem di-scFv, diabodies, tandem tri-sdcFv, minibodies, etc. In some embodiments, such as when an antibody is an scFv, a single polynucleotide of a heterologous gene encodes a single polypeptide comprising both a heavy chain and a light chain linked together. Antibody fragments also include nanobodies (e.g., sdAb, an antibody having a single, monomeric domain, such as a pair of variable domains of heavy chains, without a light chain). Multispecific antibodies (e.g., bispecific antibodies, trispecific antibodies, etc.) are known in the art and contemplated as expression products of the heterologous genes of the present invention.

In some embodiments, the heterologous gene includes a reporter sequence, which can be useful in verifying heterologous gene expression, for example, in specific cells and tissues. Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

In some embodiments, the heterologous gene does not include a coding sequence. Non-coding sequences such as shRNA, promoters, enhancers, sequences to mark DNA (e.g., for antibody recognition), PCR amplification sites, sequences that define restriction enzyme sites, site-specific recombinase recognition sites, sequences that are recognized by a protein that binds to and/or modifies nucleic acids, and linkers, may be included in the vector. In instances in which a heterologous gene is a trans-splicing molecule, non-coding sequences include binding domains that bind a target intron.

In some embodiments, the heterologous gene is from 0.1 Kb to 100 Kb in length (e.g., the heterologous gene is from 0.2 Kb to 90 Kb, from 0.5 Kb to 80 Kb, from 1.0 Kb to 70 Kb, from 1.5 Kb to 60 Kb, from 2.0 Kb to 50 Kb, from 2.5 Kb to 45 Kb, from 3.0 Kb to 40 Kb, from 3.5 Kb to 35 Kb, from 4.0 Kb to 30 Kb, from 4.5 Kb to 25 Kb, from 4.6 Kb to 24 Kb, from 4.7 Kb to 23 Kb, from 4.8 Kb to 22 Kb, from 4.9 Kb to 21 Kb, from 5.0 Kb to 20 Kb, from 5.5 Kb to 18 Kb, from 6.0 Kb to 17 Kb, from 6.5 Kb to 16 Kb, from 7.0 Kb to 15 Kb, from 7.5 Kb to 14 Kb, from 8.0 Kb to 13 Kb, from 8.5 Kb to 12.5 Kb, from 9.0 Kb to 12.0 Kb, from 9.5 Kb to 11.5 Kb, or from 10.0 Kb to 11.0 Kb in length, e.g., from 0.1 Kb to 0.5 Kb, from 0.5 Kb to 1.0 Kb, from 1.0 Kb to 2.5 Kb, from 2.5 Kb to 4.5 Kb, from 4.5 Kb to 8 Kb, from 8 Kb to 10 Kb, from 10 Kb to 15 Kb, from 15 Kb to 20 Kb in length, or greater, e.g., from 0.1 Kb to 0.25 Kb, from 0.25 Kb to 0.5 Kb, from 0.5 Kb to 1.0 Kb, from 1.0 Kb to 1.5 Kb, from 1.5 Kb to 2.0 Kb, from 2.0 Kb to 2.5 Kb, from 2.5 Kb to 3.0 Kb, from 3.0 Kb to 3.5 Kb, from 3.5 Kb to 4.0 Kb, from 4.0 Kb to 4.5 Kb, from 4.5 Kb to 5.0 Kb, from 5.0 Kb to 5.5 Kb, from 5.5 Kb to 6.0 Kb, from 6.0 Kb to 6.5 Kb, from 6.5 Kb to 7.0 Kb, from 7.0 Kb to 7.5 Kb, from 7.5 Kb to 8.0 Kb, from 8.0 Kb to 8.5 Kb, from 8.5 Kb to 9.0 Kb, from 9.0 Kb to 9.5 Kb, from 9.5 Kb to 10 Kb, from 10 Kb to 10.5 Kb, from 10.5 Kb to 11 Kb, from 11 Kb to 11.5 Kb, from 11.5 Kb to 12 Kb, from 12 Kb to 12.5 Kb, from 12.5 Kb to 13 Kb, from 13 Kb to 13.5 Kb, from 13.5 Kb to 14 Kb, from 14 Kb to 14.5 Kb, from 14.5 Kb to 15 Kb, from 15 Kb to 15.5 Kb, from 15.5 Kb to 16 Kb, from 16 Kb to 16.5 Kb, from 16.5 Kb to 17 Kb, from 17 Kb to 17.5 Kb, from 17.5 Kb to 18 Kb, from 18 Kb to 18.5 Kb, from 18.5 Kb to 19 Kb, from 19 Kb to 19.5 Kb, from 19.5 Kb to 20 Kb, from 20 Kb to 21 Kb, from 21 Kb to 22 Kb, from 22 Kb to 23 Kb, from 23 Kb to 24 Kb, from 24 Kb to 25 Kb in length, or greater, e.g., about 4.5 Kb, about 5.0 Kb, about 5.5 Kb, about 6.0 Kb, about 6.5 Kb, about 7.0 Kb, about 7.5 Kb, about 8.0 Kb, about 8.5 Kb, about 9.0 Kb, about 9.5 Kb, about 10 Kb, about 11 Kb, about 12 Kb, about 13 Kb, about 14 Kb, about 15 Kb, about 16 Kb, about 17 Kb, about 18 Kb, about 19 Kb, about 20 Kb in length, or greater).

In addition to the heterologous gene, the plasmid vectors described herein can include conventional control elements which are operably linked to the heterologous gene in a manner which permits transcription, translation, and/or expression in a target cell.

Expression control sequences include appropriate transcription initiation, termination, promoter, and enhancer sequences; efficient RNA processing signals, such as splicing and polyadenylation (polyA) signals; sequences that stabilize cytoplasmic mRNA; sequences that enhance translation efficiency (i.e., Kozak consensus sequence); sequences that enhance protein stability; and sequences that enhance secretion of the encoded product. Various expression control sequences, including promoters which are native, constitutive, inducible, and/or tissue-specific, are known in the art and may be utilized. A promoter region is operably linked to a heterologous gene if the promoter region is capable of effecting transcription of that gene such that the resulting transcript might be translated into the desired protein or polypeptide. Promoters useful as part of the vectors described herein include constitutive and inducible promoters.

Examples of promoters based on human sequences that induce retina-specific gene expression include rhodopsin kinase (GRK1) for rods and cones, PR2.1 for cones only, and RPE65 for the retinal pigment epithelium.

In some embodiments, gene expression may be achieved using a human GRK1 promoter. Thus, in certain embodiments, the promoter is a human rhodopsin kinase (GRK1) promoter.

In some embodiments, the GRK1 promoter sequence of the disclosure comprises or consists of 292 nucleotides in length and comprises or consists of nucleotides −109 to +183 of the GRK1 gene.

In some embodiments, the GRK1 promoter vector may be located in the plasmid vector upstream of the heterologous gene. For example, the GRK1 may be operably linked to the 5′ end portion of the heterologous gene.

The plasmid vector can also include a human Scaffold/Matrix Attachment Region S/MAR enhancer located in the 3′ direction downstream of the heterologous gene. S/MAR elements are used to establish long-term gene expression through the interaction with the nuclear matrix, generating chromatin loop domains that extend outwards from the heterochromatin cores. While S/MAR elements do not contain any obvious consensus or recognizable sequence, their most consistent feature appears to be an overall high A/T content, and C bases predominating on one strand (Bode J, Schlake T, RiosRamirez M, Mielke C, Stengart M, Kay V and KlehrWirth D, “Scaffold/matrix-attached regions: structural propreties creating transcriptionally active loci”, Structural and Functional Organization of the Nuclear Matrix: International Review of Citology, 162A:389453, 1995). These regions have a propensity to form bent secondary structures that may be prone to strand separation. They are often referred to as base-unpairing regions (BURs), and they contain a core-unwinding element (CUE) that might represent the nucleation point of strand separation (Benham C and al., Stress induced duplex DNA destabilization in scaffold/matrix attachment regions, J. MoL BioL, 274:181-196, 1997). Several simple AT-rich sequence motifs have often been found within MAR sequences, but for the most part, their functional importance and potential mode of action remain unclear.

A S/MAR enhancer as described herein is a nucleotide sequence sharing one or more (such as two, three or four) characteristics, such as the ones described above with a naturally occurring “S/MAR”. The S/MAR enhancer has at least one property that facilitates protein expression of any gene influenced by said S/MAR. A S/MAR enhancer has generally also the feature of being an isolated and/or purified nucleic acid preferably displaying S/MAR activity, in particular, displaying transcription modulation, preferably enhancement activity, but also displaying, e.g., expression stabilization activity and/or other activities.

The S/MAR enhancer can be inserted downstream of the promoter region to which a heterologous gene is or can be operably linked. However, in certain embodiments, it is advantageous that a S/MAR enhancer is located upstream as well as downstream or just downstream of a heterologous gene sequence of interest. Other multiple S/MAR arrangements both in cis and/or in trans are also within the scope of the present disclosure.

For heterologous genes encoding proteins, a human polyadenylation (polyA) signal sequence can be inserted following or downstream of the heterologous gene. The polyadenylation signal sequence, for example, placed 3′ of a heterologous gene, enables host factors to add a polyadenosine (polyA) tail to the end of the nascent mRNA during transcription. The polyA tail is a stretch of up to 300 adenosine ribonucleotides which protects mRNA from enzymatic degradation and also aids in translation. Accordingly, the plasmid vectors described herein may include a polyA signal sequence such as the human beta globin or rabbit beta globin polyA signals, the simian virus 40 (SV40) early or late polyA signals, the human insulin polyA signal, or the bovine growth hormone polyA signal. In one embodiment, the polyA signal sequence is a human beta globin polyA signal.

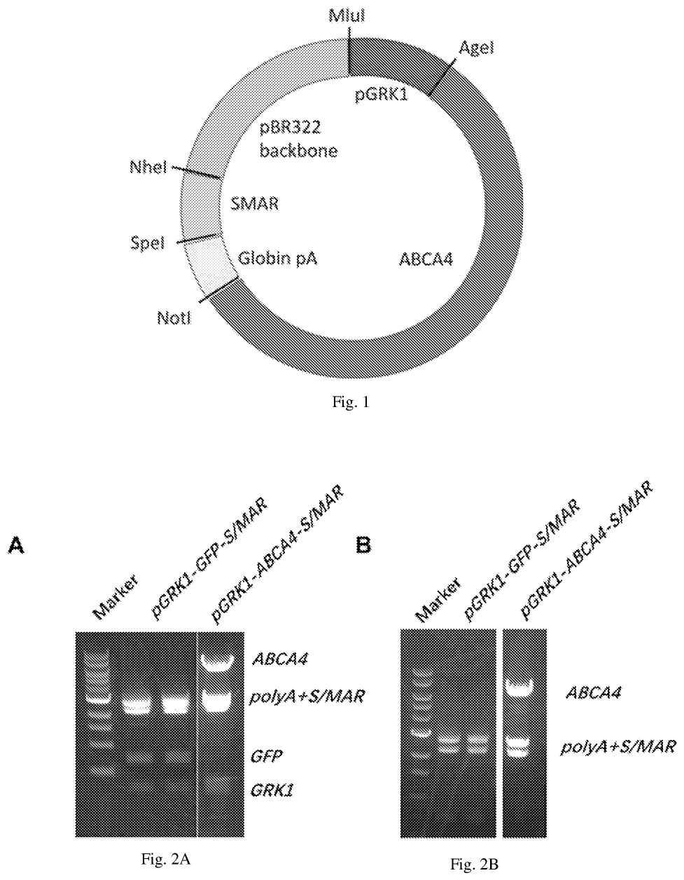

As illustrated in FIG. 1, the GRK1 promoter, heterologous gene, polyA signal sequence, and S/MAR enhancer may be cloned using an appropriately added restriction enzyme site on the plasmid vector in the order of the GRK1 promoter in the 5′ direction upstream the heterologous gene, and a polyA signal sequence and S/MAR enhancer in the 3′ direction downstream from the heterologous gene, or in the opposite order. For example, the plasmid vector may include operatively linked in a 5′ to 3′ direction: (i) a human GRK1 promoter, (ii) a heterologous gene, (iii) a polyadenylation site, and (iv) a S/MAR enhancer.

In some embodiments, the plasmid vectors provided herein include terminal repeat sequences, which may be derived, e.g., from ITRs, LTRs, or other terminal structures, e.g., as a result of circularization. The terminal repeat sequence can be at least 10 base pairs (bp) in length (e.g., from 10 bp to 500 bp, from 12 bp to 400 bp, from 14 bp to 300 bp, from 16 bp to 250 bp, from 18 bp to 200 bp, from 20 bp to 180 bp, from 25 bp to 170 bp, from 30 bp to 160 bp, or from 50 bp to 150 bp, e.g., from 10 bp to 15 bp, from 15 bp to 20 bp, from 20 bp to 25 bp, from 25 bp to 30 bp, from 30 bp to 35 bp, from 35 bp to 40 bp, from 40 bp to 45 bp, from 45 bp to 50 bp, from 50 bp to 55 bp, from 55 bp to 60 bp, from 60 bp to 65 bp, from 65 bp to 70 bp, from 70 bp to 80 bp, from 80 bp to 90 bp, from 90 bp to 100 bp, from 100 bp to 150 bp, from 150 bp to 200 bp, from 200 bp to 300 bp, from 300 bp to 400 bp, or from 400 bp to 500 bp, e.g., 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 101 bp, 102 bp, 103 bp, 104 bp, 105 bp, 106 bp, 107 bp, 108 bp, 109 bp, 110 bp, 111 bp, 112 bp, 113 bp, 114 bp, 115 bp, 116 bp, 117 bp, 118 bp, 119 bp, 120 bp, 121 bp, 122 bp, 123 bp, 124 bp, 125 bp, 126 bp, 127 bp, 128 bp, 129 bp, 130 bp, 131 bp, 132 bp, 133 bp, 134 bp, 135 bp, 136 bp, 137 bp, 138 bp, 139 bp, 140 bp, 141 bp, 142 bp, 143 bp, 144 bp, 145 bp, 146 bp, 147 bp, 148 bp, 149 bp, 150 bp, or more).

In some embodiments, one or more (e.g., one, two, three, four, five, six, or more) nucleic acids overlap between two adjacent elements. For example, in some embodiments wherein the 3′-terminal one or more nucleic acids of a first element match the 5′-terminal one or more nucleic acids of a second element linked to its 3′ end, the overlapping nucleic acids need not be repeated.

The present disclosure encompasses codon optimized variants of the nucleic acid sequences described herein. Codon optimization takes advantage of redundancies in the genetic code to enable a nucleotide sequence to be altered while maintaining the same amino acid sequence of the encoded protein.

Codon optimization may be carried out to facilitate an increase or decrease in the expression of an encoded protein. This may be effected by tailoring codon usage in a nucleotide sequence to that of a specific cell type, thus taking advantage of cellular codon bias corresponding to a bias in the relative abundance of particular tRNAs in the cell type. By altering the codons in the nucleotide sequence so that they are tailored to match the relative abundance of corresponding tRNAs, it is possible to increase expression. Conversely, it is possible to decrease expression by selecting codons for which the corresponding tRNAs are known to be rare in the particular cell type. Methods for codon optimization of nucleic acid sequences are known in the art and will be familiar to a skilled person.

Other embodiments described herein relate to self-assembled nanoparticles comprising a plurality of pH sensitive multifunctional cationic lipids complexed with one or more plasmid vector(s) described herein. The pH sensitive multifunctional cationic lipids can condense plasmid vectors and deliver the plasmid vectors to cells of the eye. The pH sensitive multifunctional cationic lipids can include a protonable amino head group, which can complex with the plasmid vectors, fatty acid or lipid tails, which can participate in hydrophobic condensation, two cysteine residues capable of forming disulfide bridges via autooxidation, and an optional targeting group that can targets and/or binds to a retinal or visual protein, such as an interphotoreceptor retinoid binding protein.

The protonable amino head group can complex with the plasmid vectors to form self-assembled nanoparticles for delivery of plasmid vectors to cells of the eye. The amines in the head groups contribute to the essential pH-sensitive characteristic of the carrier system, which is important for improving endosomal escape of the nucleic acid. Greater protonation of the amino head groups can occur in the relatively acidic environment (pH=5-6) of the endosome and lysosome compartments after cellular uptake. This enhances electrostatic interactions between the cationic carriers and the anionic phospholipids of endosomal/lysosomal membranes, promoting the bilayer destabilization and nanoparticle charge neutralization events required for efficient cytosolic release of their plasmid vector payload. By affecting the number of amines, and thus overall pKa, of the cationic carrier, the choice of head group can ultimately determine the degree to which such protonation can occur. The pH-sensitive property of the carrier system is essential so that the nanoparticles do not affect the integrity of the outer cell membrane and cause cell death, but instead are able to selectively fuse with and destabilize the endosomal and lysosomal membranes.

The cysteine residues can form disulfide bridges via autooxidation and react with functional groups of other compounds, such as those containing thiol groups. Once the plasmid vector is complexed with the pH sensitive multifunctional cationic lipids to form the self-assembled nanoparticles, the thiol groups can produce disulfide (S—S) bonds or bridges by autooxidation to form oligomers and polymers or cross-linking. The disulfide bonds can stabilize the self-assembled nanoparticles of the plasmid vectors and pH sensitive multifunctional cationic lipids and help achieve release of the plasmid vector once the nanoparticle is in the cell.

For example, the cleavage of disulfide bonds in the nanoparticle in reductive cytoplasm can facilitate cytoplasm-specific release of plasmid vector. The nanoparticles comprising the pH sensitive multifunctional cationic lipids and the plasmid vectors are stable in the plasma at very low free thiol concentration (e.g., 15 μM). When the nanoparticles are incorporated into target cells, the high concentration of thiols present in the cell (e.g., cytoplasm) will reduce the disulfide bonds to facilitate the dissociation and release of the nucleic acid.

The disulfide bonds can be readily produced by reacting the same or different pH sensitive multifunctional cationic lipids before complex with the plasmid vectors or during the complex in the presence of an oxidant. The oxidant can be air, oxygen or other chemical oxidants. Depending upon the dithiol compound selected and oxidative conditions, the degree of disulfide formation can vary in free polymers or in complexes with the plasmid vectors. Thus, pH sensitive multifunctional cationic lipids including two cysteine residues are monomers, and the monomers can be dimerized, oligomerized, or polymerized depending upon the reaction conditions.

The fatty acid or lipid tails groups can participate in hydrophobic condensation and help form compact, stable nanoparticles with the plasmid vectors and introduce amphiphilic properties to facilitate pH sensitive escape of nanoparticles from endosomal and lysosomal compartments. This is particularly useful when the compounds are used as in vivo delivery devices.

In general, the transfection efficiency of nanoparticles has been shown to decrease with increasing alkyl chain length and saturation of the lipid tail groups. When saturated, shorter aliphatic chains (C12 and C14) favor higher rates of inter-membrane lipid mixing and reportedly allow for better transfection efficiencies in vitro, as compared to in vivo, whereas the opposite is true for longer chains (C16 and C18). Typically, saturated fatty acids greater than 14 carbons in length are not favorable for nucleic acid transfections due to their elevated phase transition temperature and overall less fluidity than those that are unsaturated. However, it has been discovered that there exists a limit, at which point an increase in unsaturation and lipid fluidity is inversely correlated to transfection efficiency, primarily because some degree of rigidity is required for particle stability, as evidenced by the widespread use of cholesterol in lipid nanoparticle formulations.

In some embodiments, the pH sensitive multifunctional cationic lipids can include (1-aminoethyl)iminobis[N-(oleoylcysteinyl-1-amino-ethyl)propionamide) (ECO) or an analogue or derivative thereof. Analogues and derivatives of ECO can include, for example, ECL, SCO, TCO, EHCO, SHCO which are described in U.S. Patent Application Publication Nos. 2010/0004316, 2016/0145610, and 2019/0091347, as well as U.S. Pat. No. 10,792,374 which are all incorporated by reference in their entirety. Other pH sensitive multifunctional cationic lipids are disclosed in “A novel environment-sensitive biodegradable polydisulfide with protonable pendants for nucleic acid deliver”, Lu et al., J Control Release, 120(3):250-8(2007); “New amphiphilic carriers forming pH-sensitive nanoparticles for nucleic acid delivery”, Lu et al., Langmuir, 26(17)13874-82 (2010)1 and “Design and evaluation of new-pH-sensitive amphiphilic cationic lipids for SiRNA delivery, Lu et al. J Control Release, 171(3):296-307 (2013) all of which are incorporated by reference in their entirety.

In some embodiments, the pH sensitive multifunctional cationic lipids can include a targeting group that targets and/or binds to a retinal or visual protein, such as an interphotoreceptor retinoid binding protein. The targeting group can be attached to a cysteine residue of the pH sensitive multifunctional cationic lipids by, for example, a thiol group of the cysteine residue. The targeting group can include, for example, a retinoid, such as a retinylamine (e.g., all-trans-retinylamine) or retinoid derivative, such as (1R)-3-amino-1-[3-(cyclohexylmethoxy)phenyl]propan−1-ol; hydrochloride. In some embodiments, the targeting group is all-trans-retinylamine. In other embodiments, the targeting group is (1R)-3-amino-1-[3-(cyclohexylmethoxy)phenyl]propan-1-ol; hydrochloride. In still other embodiments, the targeting group is a synthetic retinoid derivative, such as a synthetic retinoid derivative described in U.S. Pat. No. 7,951,841 or 7,982,071 and PCT/US2015/062343, all of which are incorporated by reference in their entirety.

For example, the targeting group can include a primary amine compound of formula:

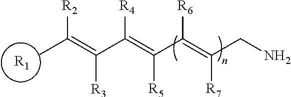

wherein R₁ is a cyclic or polycyclic ring, wherein the ring is a substituted or unsubstituted aryl, heteroaryl, cycloalkyl, or heterocyclyl;

n=1-3;

wherein R₂, R₃, R₄, R₅, R₆, and R₇, are each individually hydrogen, a substituted or unsubstituted C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms (wherein from 1-3 of the ring atoms is independently selected from N, NH, N(C₁-C₆ alkyl), NC(O)(C₁-C₆ alkyl), O, and S), C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, or phosphino or combinations thereof, wherein, R₂ and R₄ may be linked to form a cyclic or polycyclic ring, wherein the ring is a substituted or unsubstituted aryl, heteroaryl, cycloalkyl, or heterocyclyl; and pharmaceutically acceptable salts thereof.

The targeting group can be conjugated directly to the thiol group of the cysteine residue of the pH sensitive multifunctional cationic lipids or indirectly via a linker (e.g., polyethylene glycol) prior or during the formation of nanoparticles. Depending upon the selection of the targeting group, the targeting group can be covalently bonded to either the thiol group of the cysteine residues.

In one aspect, the targeting group is indirectly attached to the pH sensitive multifunctional cationic lipids by a linker. Examples of linkers include, but are not limited to, a polyamine group, a polyalkylene group, a polyamino acid group or a polyethylene glycol group. The selection of the linker as well as the molecular weight of the linker can vary depending upon the desired properties. In one aspect, the linker is polyethylene glycol having a molecular weight from 500 to 10,000, 500 to 9,000, 500 to 8,000, 500 to 7,000, or 2,000 to 5,000. In certain aspects, the targeting group is first reacted with the linker in a manner such that the targeting group is covalently attached to the linker. For example, the linker can possess one or more groups that can react with an amino group present on a targeting group. The linker also possesses additional groups that react with and form covalent bonds with the compounds described herein. For example, the linker can possess maleimide groups that readily react with the thiol groups. The selection of functional groups present on the linker can vary depending upon the functional groups present on the compound and the targeting group. In one aspect, the targeting group is a retinoid, such as a retinylamine (e.g., all-trans-retinylamine) or retinoid derivative, such as (1R)-3-amino-1-[3-(cyclohexylmethoxy)phenyl]propan-1-ol; hydrochloride, that is covalently attached to polyethylene glycol.

In some embodiment, the linker can include an acid labile bond, such as formed by incorporation of a hydrazone into the linker, that is hydrolyzable in an endolysomal environment following uptake to cells, such as retinal or retinal pigment epithelium cells. For example, the linker can be covalently linked to the compound by at least one of a covalent hydrolyzable ester, covalent hydrolyzable amide, covalent photodegradable urethane, covalent hydrolyzable ester, or covalent hydrolyzable acrylate-thiol linkage. Following cellular uptake of the compound, within the late endosomes, the increasingly acidic environment can cleave the acid labile linkage to promote shedding of a polymer linker, such as PEG, and expose the core of the compound/nucleic complex nanoparticle.

In some embodiments, the pH sensitive multifunctional cationic lipids can have formula (I):

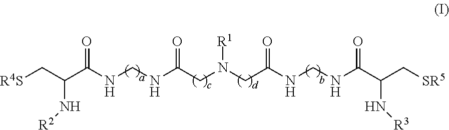

wherein R¹ is an alkylamino group or a group containing at least one aromatic group; R² and R³ are independently an aliphatic group or a hydrophobic group, derived, for example, from a fatty acid;

R⁴ and R⁵ are independently H, a substituted or unsubstituted alkyl group, an alkenyl group, an acyl group, or an aromatic group, or includes a polymer, a targeting group, or a detectable moiety; a, b, c, and d are independently an integer from 1 to 10 (e.g., a, b, c, and d are each 2); and pharmaceutically acceptable salts thereof.

In some embodiments, R¹ can include at least one of:

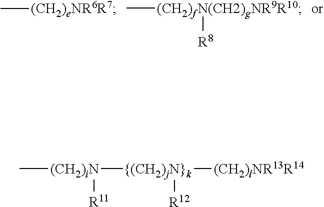

where R⁶, R⁷, R⁸, R⁹, R¹⁰, R¹¹, R¹², R¹³, and R¹⁴ are independently hydrogen, an alkyl group, a hydrophobic group, or a nitrogen containing substituent; and e, f, g, i, j, k, and 1, are an integer from 1 to 10.

For example, R¹ can include at least one of CH₂NH₂, CH₂CH₂NH₂, CH₂CH₂CH₂NH₂, CH₂CH₂CH₂CH₂NH₂, CH₂CH₂CH₂CH₂CH₂NH₂, CH₂NHCH₂CH₂CH₂NH₂,CH₂CH₂NHCH₂CH₂CH₂NH₂, CH₂CH₂CH₂NHCH₂CH₂CH₂CH₂NHCH₂CH₂CH₂NH₂, CH₂CH₂NHCH₂CH₂CH₂CH₂NH₂, CH₂CH₂NHCH₂CH₂CH₂NHCH₂CH₂CH₂HN₂, or CH₂CH₂NH(CH₂CH₂NH)_{d}CH₂CH₂NH₂, where d is from 0 to 10.

In some embodiments, R¹ can be CH₂CH₂NH₂ or CH₂CH₂NHCH₂CH₂CH₂NHCH₂CH₂CH₂HN₂.

In other embodiments, R² and R³ are independently an aliphatic group or a hydrophobic group derived from fatty acid, such as oleic acid or linoleic acid, and are the same or different. The additional double bond in linoleic acid introduces an extra kink into the hydrocarbon backbone, giving the compound a broader conical shape than oleic acid and increasing its fluidity. When incorporated into a nanoparticle structure, the extra degree of unsaturation elevates the propensity to form the hexagonal phase during an impending membrane fusion event of cellular uptake.

In some embodiments, at least one of R⁴ or R⁵ includes a retinoid, such as a retinylamine (e.g., all-trans-retinylamine) or retinoid derivative, such as (1R)-3-amino-1-[3-(cyclohexylmethoxy)phenyl]propan−1-ol; hydrochloride, that is covalently attached to a polymer linker, such as polyethylene glycol.

The pH sensitive multifunctional cationic lipids having the general formula I can be synthesized using solid phase techniques known in the art. In general, the approach involves the systematic protection/elongation/deprotection to produce a dithiol compound. The hydrophobic group is produced by reacting oleic acid with the amino group present on the cysteine residue. The targeting group is conjugated to a PEG spacer and then conjugated to the compound via a Michael addition reaction.

Any of the pH sensitive multifunctional cationic lipids described herein can exist or be converted to the salt thereof. In one aspect, the salt is a pharmaceutically acceptable salt. The salts can be prepared by treating the free acid with an appropriate amount of a chemically or pharmaceutically acceptable base. Representative chemically or pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C., such as at room temperature. The molar ratio of the compound to base used is chosen to provide the ratio desired for any particular salts. For preparing, for example, the ammonium salts of the free acid starting material, the starting material can be treated with approximately one equivalent of base to yield a salt.

In another aspect, any of the pH sensitive multifunctional cationic lipids described herein can exist or be converted to the salt with a Lewis base thereof. The compounds can be treated with an appropriate amount of Lewis base. Representative Lewis bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, THF, ether, thiol reagent, alcohols, thiol ethers, carboxylates, phenolates, alkoxides, water, and the like. In one aspect, the reaction is conducted in water, alone or in combination with an inert, water-miscible organic solvent, at a temperature of from about 0° C. to about 100° C. such as at room temperature. The molar ratio of the compound to base used is chosen to provide the ratio desired for any particular complexes. For example, the ammonium salts of the free acid starting material, the starting material can be treated with approximately one equivalent of chemically or pharmaceutically acceptable Lewis base to yield a complex.

If the pH sensitive multifunctional cationic lipids possess carboxylic acid groups, these groups can be converted to pharmaceutically acceptable esters or amides using techniques known in the art. Alternatively, if an ester is present on the dendrimer, the ester can be converted to a pharmaceutically acceptable ester using transesterification techniques.

The plasmid vectors can be complexed to the pH sensitive multifunctional cationic lipids described herein by admixing the plasmid vectors and the pH sensitive multifunctional cationic lipids. The pH of the reaction can be modified to convert the amino groups present on the pH sensitive multifunctional cationic lipids described herein to cationic groups. For example, the pH can be adjusted to protonate the amino group. With the presence of cationic groups on the pH sensitive multifunctional cationic lipids, the plasmid can electrostatically bond (i.e., complex) to the compound. In one aspect, the pH is from 6 to 7.4. In another aspect, the N/P ratio can be from 0.5 to 100, where N is the number of amino or nitrogen atoms present on the compound that can be form a positive charge and P is the number of phosphate groups present on the plasmid vector. Thus, by modifying the pH sensitive multifunctional cationic lipids with the appropriate number of amino groups in the head group, it is possible to tailor the bonding (e.g., type and strength of bond) between the plasmid vector and the pH sensitive multifunctional cationic lipids. The N/P ratio can be adjusted depending on the cell type to which the plasmid vector is to be delivered. In some embodiments where the cell is a photoreceptor, the N/P ratio can be at least about 6, at least about 10, or at least about 15. In other embodiments, the N/P ration can be from about 6 to about 20. In some embodiments, the self-assembled nanoparticles can have an amine to phosphate (N/P) ratio of about 4 to about 12, preferably, about 6 to about 10.

In some embodiments, the self-assembled nanoparticle can have a diameter of about 1000 nanometers or less, or about 50 nm to about 500 nm, about 100 nm to about 400 nm, or about 150 nm to about 300 nm.

In other aspects, the self-assembled nanoparticles described herein can be designed so that the plasmid vector escapes endosomal and/or lysosomal compartments at the endosomal-lysosomal pH. For example, the self-assembled nanoparticles can be designed such that its structure and amphiphilicity changes at endosomal-lysosomal pH (5.0-6.0) and disrupts endosomal-lysosomal membranes, which allows entry of the nanoparticle into the cytoplasm. In one aspect, the ability of specific endosomal-lysosomal membrane disruption of the self-assembled nanoparticles described herein can be tuned by modifying their pH sensitive amphiphlicity by altering the number and structure of protonatable amines and lipophilic groups. For example, decreasing the number of protonatable amino groups can reduce the amphiphilicity of a nanoparticle produced by the pH sensitive multifunctional cationic lipids at neutral pH. In one aspect, the pH sensitive multifunctional cationic lipids described herein have 1 to 50, 1 to 40, 1 to 30, 1 to 20, 1 to 10, 1 to 8, 1 to 6, 1 to 4, or 2 protonatable amino or substituted amino groups. The pH-sensitive amphiphilicity of the pH sensitive multifunctional cationic lipids and nanoparticles produced by the pH sensitive multifunctional cationic lipids can be used to fine-tune the overall pKa of the nanoparticle. Low amphiphilicity of the nanoparticles at physiological pH can minimize non-specific cell membrane disruption and nonspecific tissue uptake of the nucleic acid/MFC system. In certain aspects, it is desirable that the carriers have low amphiphilicity at the physiological pH and high amphiphilicity at the endosomal-lysosomal pH, which will only cause selective endosomal-lysosomal membrane disruption with the nanoparticles.

Optionally, the surface of the nanoparticle complexes can be modified by, for example, covalently incorporating polyethylene glycol by reacting unpolymerized free thiol of the nanoparticle to reduce non-specific tissue uptake in vivo. For example, PEG-maleimide reacts rapidly with free thiol groups. The molecular weight of the PEG can vary depending upon the desired amount of hydrophilicity to be imparted on the carrier. PEG-modification of the carrier can also protect nanoparticles composed of the nucleic acid from enzymatic degradation upon uptake by the cell (e.g., endonucleases).

In some embodiments, the amount or mole percent of the targeting groups provided on or attached to the surface of the nanoparticle can be about 1 mol % to about 10 mol % of the compounds that form the nanoparticle, for example, about 1 mol % to about 5 mol % (e.g., about 2.5 mol %) of the compounds that form the nanoparticle.

Advantageously, multifunctional pH-sensitive carriers formed using the compounds have improved stability when administered systemically to a subject, protect condensed plasmid vectors from degradation, and promote endosomal escape and cytosolic release upon cellular uptake.

The plasmid vectors and/or self-assembled nanoparticles may be formulated into pharmaceutical compositions. These compositions may comprise, in addition to the plasmid vectors and/or self-assembled nanoparticles, a pharmaceutically acceptable carrier, diluent, excipient, buffer, stabilizer or other materials well known in the art. Such materials should be non-toxic and should not interfere with the efficacy of the plasmid vectors and/or self-assembled nanoparticles. The precise nature of the carrier or other material may be determined by the skilled person according to the route of administration, e.g., subretinal, direct retinal, suprachoroidal or intravitreal injection.

The pharmaceutical composition may be in liquid form. Liquid pharmaceutical compositions include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included.

For injection at the site of affliction, the pharmaceutical composition may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection or TMN 200. Preservatives, stabilisers, buffers, antioxidants and/or other additives may be included as required.

Buffers may have an effect on the stability and biocompatibity of the plasmid vectors and nanoparticles described herein following storage and passage through injection devices for gene therapy. In some embodiments, the plasmid vectors and/or self-assembled nanoparticles described herein may be diluted in TMN 200 buffer to maintain biocompatibility and stability. TMN 200 buffer comprises 20 mM Tris (pH adjusted to 8.0), 1 mM MgCl₂ and 200 mM NaCl at pH 8.

In some embodiments, the pharmaceutical composition can include an amount sucrose effective to enhance the stability of the self-assembled nanoparticles under different or differing storage temperatures. The different or differing storage temperatures can range from about −20° C. to about 4° C. For example, the pharmaceutical composition can include about 5% to about 20% sucrose.

In some embodiments, pharmaceutical composition can have a substantially neutral pH. The substantially neutral pH is about 6.0 to about 8.0, about 6.2 to about 7.8, about 6.5 to about 7.5, about 6.8 to about 7.2, or about 7.

In some embodiments, the pharmaceutical composition is free of an excipient besides sucrose.

In other embodiments, the pharmaceutical composition includes a concentration of self-assembled nanoparticles of about 50 ng/μl to about 500 ng/μl, about 100 ng/μl to about 300 ng/μl, or about 150 ng/μl to about 250 ng/μl.

For delayed release, the plasmid vectors and/or self-assembled nanoparticles may optionally be included in a pharmaceutical composition which is formulated for slow release, such as in microcapsules formed from biocompatible polymers according to methods known in the art.

The plasmid vectors, the self-assembled nanoparticles, or the pharmaceutical composition described herein can be used in a method of inducing episomal expression of a heterologous gene in a subject in need thereof. The method generally involves contacting the cell with plasmid vectors, the self-assembled nanoparticles, or the pharmaceutical composition described herein wherein the plasmid vector is taken up into the cell. In one aspect, the plasmid vectors, the self-assembled nanoparticles, or the pharmaceutical composition described herein can facilitate the delivery of plasmid vector as therapy for genetic disease by supplying deficient or absent gene products to treat any genetic disease or by silencing gene expression. Techniques known in the art can be used to measure the efficiency of the compounds described herein to deliver nucleic acids to a cell.

In some embodiments, the target cell can be a cell within the eye. Examples of cells within the eye can include cells located in the ganglion cell layer (GCL), the inner plexiform layer inner (IPL), the inner nuclear layer (INL), the outer plexiform layer (OPL), outer nuclear layer (ONL), outer segments (OS) of rods and cones, the retinal pigmented epithelium (RPE), the inner segments (IS) of rods and cones, the epithelium of the conjunctiva, the iris, the ciliary body, the corneum, and epithelium of ocular sebaceous glands.

Advantageously, the plasmid vectors, the self-assembled nanoparticles, or the pharmaceutical composition provide surprisingly high levels of expression of full-length retinal proteins, such as ABCA4, in transduced cells, with limited production of unwanted truncated fragments of the retinal proteins.

In some embodiments, the plasmid vectors or the self-assembled nanoparticles described herein can increase the expression of a visual cycle protein, such as ABCA4 or RPE65, at an amount effective to enhance vision and/or restore normal vision. In certain embodiments of any of the foregoing methods, the the plasmid vectors or the self-assembled nanoparticles described herein can increase the expression of a visual cycle protein (e.g., ABCA4 or RPE65) associated with a nonsense or missense mutation of an inheritable retinal disorder (IRD) (e.g., Stargardt disease or LCA). For example, in certain embodiments, a cell contains about 5%, about 10%, about 20%, about 30%, about 40%, about 50%, about 60%, about 70%, about 80%, or about 90% of the gene product relative to a cell without the missense or nonsense mutation. In certain embodiments, the cell contains from about 5% to about 80%, about 5% to about 60%, about 5% to about 40%, about 5% to about 20%, about 5% to about 10%, about 10% to about 80%, about 10% to about 60%, about 10% to about 40%, about 10% to about 20%, about 20% to about 80%, about 20% to about 60%, about 20% to about 40%, about 40% to about 80%, about 40% to about 60%, or about 60% to about 80% of the gene product relative to a cell without the missense or nonsense mutation. In certain embodiments, there is no detectable gene product in the cell. Gene product amount or expression may be measured by any method known in the art, for example, Western blot or ELISA.

In certain embodiments, wherein the gene is an IRD related gene (e.g., ABCA4 or RPE65 gene) with a nonsense mutation that encodes a visual cycle protein, the plasmid vectors or the self-assembled nanoparticles described herein can be selected to increase the visual cycle protein (e.g., ABCA4 or RPE65) expression in a cell by at least about 4%, about 5%, about 6%, about 7%, about 8%, about 9%, about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, about 500%, about 600%, about 700%, about 800%, about 900%, or about 1000% relative to a cell, tissue, or subject without the nonsense mutation.

In certain embodiments, the plasmid vectors or the self-assembled nanoparticles described herein can be selected that increase visual cycle protein (e.g., ABCA4 or RPE65) expression in a cell from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a cell, tissue, or subject with the RPE65 mutation.

In certain embodiments, where the gene is an ABCA4 gene with a nonsense mutation, the plasmid vectors or the self-assembled nanoparticles described herein can increase ABCA4 expression in a retina cell or retinal pigment epithelium cell by at least about 4%, about 5%, about 6%, about 7%, about 8%, about 9%, about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, about 500%, about 600%, about 700%, about 800%, about 900%, or about 1000% relative to a retina cell or retinal pigment epithelium cell without the nonsense mutation.

In certain embodiments, the method increases ABCA4 expression in a retina cell or retinal pigment epithelium cell by from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a retina cell or retinal pigment epithelium cell with the ABCA4 mutation.

With an optimized recombination, the full length protein ABCA4 can be expressed in a photoreceptor outer segments in subjects with mutant retinal proteins and at levels sufficient to reduce bisretinoid formation and correct the autofluorescent phenotype on retinal imaging. These observations support a plasmid vector approach for gene therapy to treat Stargardt disease.

Stargardt disease resulting from mutations in the ABCA4 gene is the most common inherited macular dystrophy, affecting 1 in 8,000-10,000 people and resulting from mutations in the ABCA4 gene. ABCA4 mutations are responsible for Stargardt disease and other cone and cone-rod dystrophies. Stargardt disease resulting from mutations in the ABCA4 gene is the most common cause of blindness in children in the developed world. The disease often presents in childhood and becomes progressively worse over the course of a patient's lifetime therefore therapeutic intervention at any point could prevent or slow further sight loss. This disease is progressive, and often becomes symptomatic in childhood but after the period of visual development, which provides ample opportunity for therapeutic intervention to prevent or slow further sight loss.

ABCA4 clears toxic metabolites from the photoreceptor outer segments discs. The absence of functional ABCA4 leads to photoreceptor degeneration. Photoreceptor outer segment discs comprise the light sensing protein rhodopsin and the transmembrane protein ABCA4. ABCA4 controls the export of certain toxic visual cycle byproducts. Visual pigments comprise an opsin and a chromophore, for example a retinoid such as I1-cis-retinal. In the visual cycle, sometimes termed the retinoid cycle, retinoids are bleached and recycled between the photoreceptors and the retinal pigment epithelium (RPE). Upon activation of rhodopsin during phototransduction, 11-cis-retinal is isomerized to all-trans-retinal, which dissociates from the opsin. All-trans-retinal is transported to the RPE, and either stored or converted back to 11-cis-retinal and transported back to photoreceptors to complete the visual cycle.

Mutations in ABCA4 prevent the transport of retinoids from photoreceptor cell disc outer membranes to the retinal pigment epithelium (RPE), which leads to a build-up of undesired retinoid derivatives in the photoreceptor outer segments. Due to constant generation of photoreceptor outer segments, as older discs become more terminal they are consumed by the RPE. In photoreceptor cells carrying mutant, non-functional ABCA4, bisretinoids retained in the disc membranes build up in the RPE cells with further biochemical processes taking place that lead to formation of the toxicity compound A2E, a key element of lipofuscin. ABCA4 mutations are associated with the build-up of toxins in the photoreceptors and the RPE. Exemplary toxins include, but are not limited to, all-trans-retinal, bisretinoids and lipofuscin.

Lack of functional ABCA4 prevents the transport of free retinaldehyde from the luminal to the cytoplasmic side of the photoreceptor cell disc outer membranes, resulting in increased formation, or amplifying, the formation of retinoid dimers (bisretinoids). Upon daily phagocytosis of the distal outer segments of photoreceptor cells by the retinal pigment epithelium (RPE), the retinoid derivatives are processed further, leading to accumulation of bisretinoids. The retinoid derivatives are processed but are insoluble and accumulate. The outcome of this accumulation leads to dysfunction and eventual death of the RPE cells with subsequent secondary loss of the overlying photoreceptors through degeneration and subsequent death.

In some embodiments, the plasmid vectors, the self-assembled nanoparticles, or the pharmaceutical composition generates a full length ABCA4 transgene in one or more cells of an eye of subject. In some embodiments, the subject has Stargardt disease. In some embodiments, the one or more cells comprise photoreceptor cells. In some embodiments, the one or more cells comprise RPE cells. In some embodiments, the one or more cells comprise RPE cells, photoreceptor cells, or a combination thereof.

In some embodiments, expression of the ABCA4 gene in the one or more cells of the eye of the subject slows the degeneration of photoreceptor cells. In some embodiments, the one or more cells comprise RPE cells, photoreceptor cells, or a combination thereof. In some embodiments, expression of the ABCA4 transgene in the one or more cells of the eye of the subject prevents the death of photoreceptor cells. In some embodiments, expression of the ABCA4 transgene in the one or more cells of the eye of the subject prevents the degeneration of photoreceptor cells. In some embodiments, expression of the ABCA4 transgene in the one or more cells of the eye of the subject restores the photoreceptor cells to healthy or viable photoreceptor cells.

In some embodiments, expression of the ABCA4 transgene in the one or more cells of the eye of the subject prevents the death of RPE cells. In some embodiments, expression of the ABCA4 transgene in the one or more cells of the eye of the subject prevents the death of RPE cells and the degeneration of photoreceptor cells.

In some embodiments, a method for expressing a human ABCA4 protein in a target cell includes administering to the subject the plasmid vectors, the self-assembled nanoparticles, or the pharmaceutical composition described herein to the eye of the subject, wherein the plasmid vector transduce the target cell such that a functional ABCA4 protein is expressed in the target cell.

In some embodiments, the plasmid vectors, the self-assembled nanoparticles, or the pharmaceutical composition described herein may be administered to the eye of a subject by subretinal, direct retinal, suprachoroidal or intravitreal injection.

A skilled person will be familiar with and well able to carry out individual subretinal, direct retinal, suprachoroidal or intravitreal injections.

Subretinal injections are injections into the subretinal space, i.e., underneath the neurosensory retina. During a subretinal injection, the injected material is directed into, and creates a space between, the photoreceptor cell and retinal pigment epithelial (RPE) layers.

When the injection is carried out through a small retinotomy, a retinal detachment may be created. The detached, raised layer of the retina that is generated by the injected material is referred to as a “bleb”.

The hole created by the subretinal injection may be sufficiently small that the injected solution does not significantly reflux back into the vitreous cavity after administration. Such reflux would be problematic when a medicament is injected, because the effects of the medicament would be directed away from the target zone. Preferably, the injection creates a self-sealing entry point in the neurosensory retina, i.e., once the injection needle is removed, the hole created by the needle reseals such that very little or substantially no injected material is released through the hole.

To facilitate this process, specialist subretinal injection needles are commercially available (e.g., DORC 41G Teflon subretinal injection needle, Dutch Ophthalmic Research Center International BV, Zuidland, The Netherlands). These are needles designed to carry out subretinal injections.

In some embodiments, subretinal injection comprises a scleral tunnel approach through the posterior pole to the superior retina with a Hamilton syringe and 34-gauge needle (ESS labs, UK). Alternatively, or in addition, subretinal injections can comprise performing an anterior chamber paracentesis with a 33G needle prior to the sub-retinal injection using a WPI syringe and a bevelled 35G-needle system (World Precision Instruments, UK).

Animal subjects, can be anaesthetized, for example, by intraperitoneal injection containing ketamine (80 mg/kg) and xylazine (10 mg/kg) and pupils fully dilated with tropicamide eye drops (Mydriaticum 1%, Bausch & Lomb, UK) and phenylephrine eye drops (phenylephrine hydrochloride 2.5%, Bausch & Lomb, UK). Proxymetacaine eye drops (proxymetacaine hydrochloride 0.5%, Bausch & Lomb, UK) can also be applied prior to sub-retinal injection. Post-injection, chloramphenicol eye drops can be applied (chloramphenicol 0.5%, Bausch & Lomb, UK), anaesthesia reversed with atipamezole (2 mg/kg), and carbomer gel applied (Viscotears, Novartis, UK) to prevent cataract formation. Unless damage to the retina occurs during the injection, and as long as a sufficiently small needle is used, injected material remains localized between the detached neurosensory retina and the RPE at the site of the localized retinal detachment (i.e., does not reflux into the vitreous cavity). Indeed, the persistence of the bleb over a short time frame indicates that there may be little escape of the injected material into the vitreous. The bleb may dissipate over a longer time frame as the injected material is absorbed.

Visualizations of the eye, for example the retina, for example using optical coherence tomography, may be made pre-operatively.

In some embodiments, the plasmid vectors, self-assembled nanoparticles, or the pharmaceutical composition described may be delivered with accuracy and safety by using a two-step method in which a localized retinal detachment is created by the subretinal injection of a first solution. The first solution does not comprise the vector. A second subretinal injection is then used to deliver the medicament comprising the plasmid vectors, self-assembled nanoparticles, or the pharmaceutical composition into the subretinal fluid of the bleb created by the first subretinal injection. Because the injection delivering the plasmid vectors, self-assembled nanoparticles, or the pharmaceutical composition is not being used to detach the retina, a specific volume of solution may be injected in this second step.

The volume of solution injected to at least partially detach the retina may be, for example, about 10-1000 μL, for example about 50-1000, 100-1000, 250-1000, 500-1000, 10-500, 50-500, 100-500, 250-500 μL. The volume may be, for example, about 10, 50, 100, 200, 300, 400, 500, 600, 700, 800, 900 or 1000 μL.

The volume of the pharmaceutical composition injected may be, for example, about 0.1-500 μL, for example about 0.5-500, 1-500, 2-500, 3-500, 4-500, 5-250, 1-250, 2-250 or 5-150 μL. In other embodiments, the volume of the pharmaceutical composition administered to the subject is about 0.1 μL to about 2 μL, about 0.2 μL to about 1.5 μL, or about 0.5 μL to about 1 μL.

In some embodiments, the concentration of the self-assembled nanoparticles in the pharmaceutical composition is about 50 ng/μl to about 500 ng/μl, about 100 ng/μl to about 300 ng/μl, or about 150 ng/μl to about 250 ng/μl.

In some embodiments, for example during end-stage retinal degenerations, identifying the retina is difficult because it is thin, transparent and difficult to see against the disrupted and heavily pigmented epithelium on which it sits. The use of a blue vital dye may facilitate the identification of the retinal hole made for the retinal detachment procedure so that the pharmaceutical composition can be administered through the same hole without the risk of reflux back into the vitreous cavity.

The use of the blue vital dye may also identify any regions of the retina where there is a thickened internal limiting membrane or epiretinal membrane, as injection through either of these structures may hinder clean access into the subretinal space. Furthermore, contraction of either of these structures in the immediate post-operative period may lead to stretching of the retinal entry hole, which may lead to reflux of the medicament into the vitreous cavity.

In some embodiments, the plasmid vectors, the self-assembled nanoparticles, or the pharmaceutical composition described herein may be administered via suprachoroidal injection. Any means of suprachoroidal injection is envisaged as a potential delivery system for the plasmid vectors, the self-assembled nanoparticles, or the pharmaceutical composition described herein. Suprachoroidal injections are injections into the suprachoroidal space, which is the space between the choroid and the sclera. Injection into the suprachoroidal space is thus a potential route of administration for the delivery of compositions to proximate eye structures such as the retina, retinal pigment epithelium (RPE) or macula. In some embodiments, injection into the suprachoroidal space is done in an anterior portion of the eye using a microneedle, microcannula, or microcatheter. An anterior portion of the eye may comprise or consist of an area anterior to the equator of the eye. The plasmid vectors or the self-assembled nanoparticles described herein may diffuse posteriorly from an injection site via a suprachoroidal route. In some embodiments, the suprachoroidal space in the posterior eye is injected directly using a catheter system. In this embodiment, the suprachoroidal space may be catheterized via an incision in the pars plana. In some embodiments, an injection or an infusion via a suprachoroidal route traverses the choroid, Bruch's membrane and/or RPE layer to deliver the plasmid vectors, the self-assembled nanoparticles, or the pharmaceutical composition described herein to a subretinal space. In some embodiments, including those in which the plasmid vectors, the self-assembled nanoparticles, or the pharmaceutical composition described herein is delivered to a subretinal space via a suprachoroidal route, one or more injections is made into at least one of the sclera, the pars plana, the choroid, the Bruch's membrane, and the RPE layer. In some embodiments, including those in which the plasmid vectors, the self-assembled nanoparticles, or the pharmaceutical composition described herein is delivered to a subretinal space via a suprachoroidal route, a two-step procedure is used to create a bleb in a suprachoroidal or a subretinal space prior to delivery of the plasmid vectors, the self-assembled nanoparticles, or the pharmaceutical composition described herein.

In some embodiments, the plasmid vectors, the self-assembled nanoparticles, or the pharmaceutical composition is administered repeatedly.

In other embodiments, the plasmid vectors, the self-assembled nanoparticles, or the pharmaceutical composition is administered in a single dose.

In some embodiments, the plasmid vectors, the self-assembled nanoparticles, or the pharmaceutical composition can be effective in treating the subject's visual function. The visual function can be assessed by microperimetry, dark-adapted perimetry, assessment of visual mobility, visual acuity, ERG, or reading assessment.

The baseline or improved visual acuity of a subject may be measured by having the subject navigate through an enclosure characterized by low light or dark conditions and including one or more obstacles for the subject to avoid. The subject may be in need of a composition of the disclosure, optionally, provided by a method of treating of the disclosure. The subject may have received the plasmid vectors, the self-assembled nanoparticles, or the pharmaceutical composition, optionally, provided by a method of treating in one or both eyes and in one or more doses and/or procedures/injections. The enclosure may be indoors or outdoors. The enclosure is characterized by a controlled light level ranging from a level that recapitulates daylight to a level that simulates complete darkness. Within this range, the controlled light level of the enclosure may be preferably set to recapitulate natural dusk or evening light levels at which a subject of the disclosure prior to receiving a composition of the disclosure may have decreased visual acuity. Following administration of a composition of the disclosure, the subject may have improved visual acuity at all light levels, but the improvement is preferably measured at lower light levels, including those that recapitulate natural dusk or evening light levels (indoors or outdoors).

In some embodiments of the enclosure, the one or more obstacles are aligned with one or more designated paths and/or courses within the enclosure. A successful passage through the enclosure by a subject may include traversing a designated path and avoiding traversal of a non-designated path. A successful passage through the enclosure by a subject may include traversing any path, including a designated path, while avoiding contact with one or more obstacles positioned either within a path or in proximity to a path. A successful or improved passage through the enclosure by a subject may include traversing any path, including a designated path, while avoiding contact with one or more obstacles positioned either within a path or in proximity to a path with a decreased time required to traverse the path from a designated start position to a designated end position (e.g., when compared to a healthy individual with normal visual acuity or when compared to a prior traversal by the subject). In some embodiments, an enclosure may comprise at least 1, 2, 3, 4, 5, 6, 7, 8, 9 or 10 paths or designated paths. A designated path may differ from a non-designated path by the identification of the designated path by the experimenter as containing an intended start position and an intended end position.

In some embodiments of the enclosure, the one or more obstacles are not fixed to a surface of the disclosure. In some embodiments, the one or more obstacles are fixed to a surface of the disclosure. In some embodiments, the one or more obstacles are fixed to an internal surface of the enclosure, including, but not limited to, a floor, a wall and a ceiling of the enclosure. In some embodiments, the one or more obstacles comprise a solid object. In some embodiments, the one or more obstacles comprise a liquid object (e.g., a “water hazard”). In some embodiments, the one or more obstacles comprise in any combination or sequence along at least one path or in close proximity to a path, an object to be circumvented by a subject; an object to be stepped over by a subject; an object to be balanced upon by walking or standing; an object having an incline, a decline or a combination thereof; an object to be touched (for example, to determine a subject's ability to see and/or judge depth perception); and an object to be traversed by walking or standing beneath it (e.g., including bending one or more directions to avoid the object). In some embodiments of the enclosure, the one or more obstacles must be encountered by the subject in a designated order.

In certain embodiments, baseline or improved visual acuity of a subject may be measured by having the subject navigate through a course or enclosure characterized by low light or dark conditions and including one or more obstacles for the subject to avoid, wherein the course or enclosure is present in an installation. In particular embodiments, the installation includes a modular lighting system and a series of different mobility course floor layouts. In certain embodiments, one room houses all mobility courses with one set of lighting rigs. For example, a single course may be set up at a time during mobility testing, and the same room/lighting rigs may be used for mobility testing independent of the course (floor layout) in use. In particular embodiments, the different mobility courses provided for testing are designed to vary in difficulty, with harder courses featuring low contrast pathways and hard to see obstacles, and easier courses featuring high contrast pathways and easy to see obstacles.

In some embodiments of the enclosure, the subject may be tested prior to administration of the plasmid vectors, the self-assembled nanoparticles, or the pharmaceutical composition to establish, for example, a baseline measurement of accuracy and/or speed or to diagnose a subject as having a retinal disease or at risk of developing a retinal disease. In some embodiments, the subject may be tested following administration of the plasmid vectors, the self-assembled nanoparticles, or the pharmaceutical composition to determine a change from a baseline measurement or a comparison to a score from a healthy individual (e.g., for monitoring/testing the efficacy of the composition to improve visual acuity).

The baseline or improved measurement of retinal cell viability of a subject may also be measured by one or more AOSLO techniques. Scanning Laser Ophthalmoscopy (SLO) may be used to view a distinct layer of a retina of an eye of a subject. Preferably, adaptive optics (AO) are incorporated in SLO (AOSLO), to correct for artifacts in images from SLO alone typically caused by structure of the anterior eye, including, but not limited to the cornea and the lens of the eye. Artifacts produced by using SLO alone decrease resolution of the resultant image. Adaptive optics allow for the resolution of a single cell of a layer of the retina and detect directionally backscattered light (waveguided light) from normal or intact retinal cells (e.g., normal or intact photoreceptor cells).

In some embodiments of the disclosure, using an AOSLO technique, an intact cell can produce a waveguided and/or detectable signal. In some embodiments a non-intact cell does not produce a waveguided and/or detectable signal.

AOSLO may be used to image and, preferably, evaluate the retina or a portion thereof in a subject. In some embodiments, the subject has one or both retinas imaged using an AOSLO technique. In some embodiments, the subject has one or both retinas imaged using an AOSLO technique prior to administration of a composition of the disclosure (e.g., to determine a baseline measurement for subsequent comparison following treatment and/or to determine the presence and/or the severity of retinal disease). In some embodiments, the subject has one or both retinas imaged using an AOSLO technique following an administration of a composition of the disclosure (e.g., to determine an efficacy of the composition and/or to monitor the subject following administration for improvement resulting from treatment).

In some embodiments, the retina is imaged by either confocal or non-confocal (split-detector) AOSLO to evaluate a density of one or more retinal cells. In some embodiments, the one or more retinal cells include, but are not limited to a photoreceptor cell. In some embodiments, the one or more retinal cells include, but are not limited to a cone photoreceptor cell. In some embodiments, the one or more retinal cells include, but are not limited to a rod photoreceptor cell. In some embodiments, the density is measured as number of cells per millimeter. In some embodiments, the density is measured as number of live or viable cells per millimeter. In some embodiments, the density is measured as number of intact cells per millimeter (cells comprising the plasmid vectors or the self-assembled nanoparticles). In some embodiments, the density is measured as number of responsive cells per millimeter. In some embodiments, a responsive cell is a functional cell.

In some embodiments, AOSLO may be used to capture an image of a mosaic of photoreceptor cells within a retina of the subject. In some embodiments, the mosaic includes intact cells, non-intact cells or a combination thereof. In some embodiments, a mosaic comprises a composite or montage of images representing an entire retina, an inner segment, an outer segment, or a portion thereof. In some embodiments, the image of a mosaic comprises a portion of a retina comprising or contacting the plasmid vectors or the self-assembled nanoparticles. In some embodiments, the image of a mosaic comprises a portion of a retina juxtaposed to a portion of the retina comprising or contacting the plasmid vectors or the self-assembled nanoparticles. In some embodiments, the image of a mosaic comprises a treated area and an untreated area, wherein the treated area comprises or contacts the plasmid vectors or the self-assembled nanoparticles and the untreated area does not comprise or contact the plasmid vectors or the self-assembled nanoparticles.

In some embodiments, AOSLO may be used alone or in combination with optical coherence tomography (OCT) to visualize directly a retinal, a portion of a retinal or a retinal cell of a subject. In some embodiments, adaptive optics may be used in combination with OCT (AO-OCT) to visualize directly a retinal, a portion of a retinal or a retinal cell of a subject.

In some embodiments of the disclosure, the outer or inner segment is imaged by either confocal or non-confocal (split-detector) AOSLO to evaluate a density of cells therein or a level of integrity of the outer segment, the inner segment or a combination thereof. In some embodiments, AOSLO may be used to detect a diameter of an inner segment, an outer segment or a combination thereof.

The function and advantage of these and other embodiments described herein will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### Example

Plasmid Construction: ABCA4 Plasmid DNA with the Human GRK1 Promoter and Human polyA and Human S/MAR Enhancer for Treating Stargardt Disease

A new ABCA4 plasmid DNA with a human promoter and enhancer has been constructed to promote specific expression of ABCA4 in both cone and rod photoreceptors for clinical use. DNA fragment of human GRK1 (˜109 to +183) promoter was amplified from human genomic DNA according to previous publication “A photoreceptor enhancer upstream of the GRK1 gene”, IOVS, 2003. Human beta-Globin polyA was amplified from human genomic DNA as well. The human enhancer S/MAR DNA was also used in the plasmid construct to enhance gene expression. The whole plasmid map was shown in FIG. 1. The sequence was analyzed based on pEPI plasmid sequence deposited into Pubmed database. GRK1 promoter was inserted into MluI and AgeI restriction sites. S/MAR DNA was inserted into NotI and NheI restriction sites. Finally, Globin polyA DNA was inserted by using NEB HiFi assembly cloning kit due to the overlapping sequence upstream and downstream of the inserting sites of the designed primers. At the same time a SpeIrestriction site was introduced between polyA and S/MAR.

The plasmid structure was confirmed by agarose gel electrophoresis of selective digestion reactions at different restriction sites, which was shown in FIG. 2. Four restriction enzymes of MluI, AgeI, NotI and NheI were used in multiple enzyme digestion reactions. Agarose gel (1%) was prepared to separate 4 DNA fragments after digestion. In control GFP plasmid, promoter GRK1 (400 bp), GFP (700 bp), polyA+S/MAR (2.9 kb) and vector backbone (˜2.5 kb) were separated after digestion and gel running. In ABCA4 plasmid, ABCA4 fragment (6.8 kb) was identified (FIG. 2A). After a longer run of the same agarose gel, a separation of the backbone and pA+SMAR fragments were observed (FIG. 2B). These results confirmed the success of plasmid construction. Full length sequencing was also performed on the new ABCA4 plasmid. The results were in the Sequence.

### Preparation of ECO/pGRK1-ABCA4-S/MAR Nanoparticles

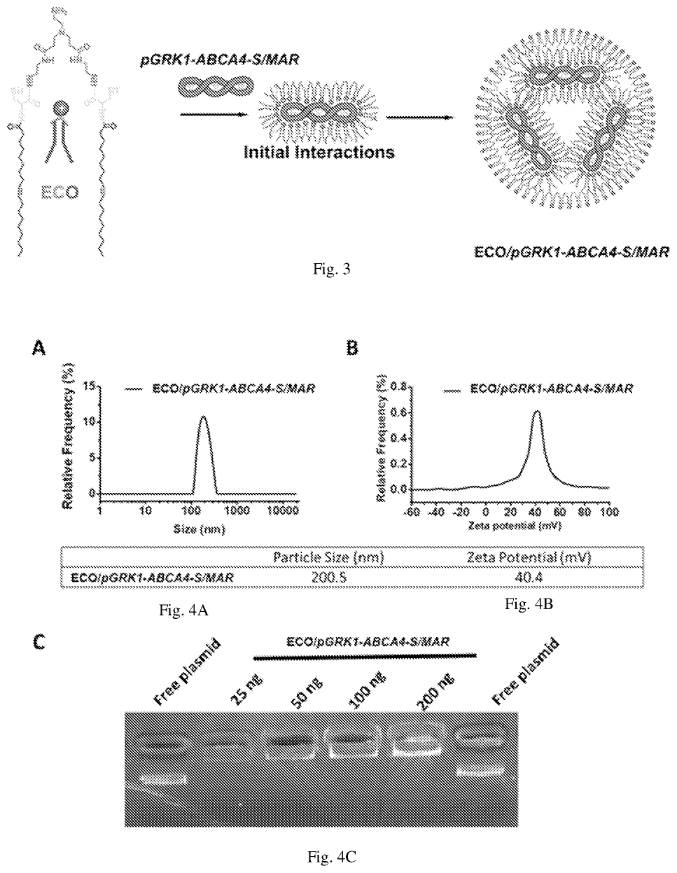

ECO/pGRK1-ABCA4-S/MAR nanoparticles were prepared through self-assembly of ECO and pGRK1-ABCA4-S/MAR in aqueous solution as demonstrated in FIG. 3. Specifically, ECO stock solution (25 mM in ethanol) and pGRK1-ABCA4-S/MAR stock solution (0.5 μg/μL) were mixed and shaken in an aqueous solution at predetermined concentrations (25, 50, 100, or 200 ng/μL), and N/P ratios (6, 8 or 10) for 30 min at room temperature to give pGRK1-ABCA4-S/MAR nanoparticles.

### Characterization of ECO/pGRK1-ABCA4-S/MAR Nanoparticles

ECO/pGRK1-ABCA4-S/MAR nanoparticles were formulated at various N/P ratios (6, 8 and 10) with different DNA concentrations (25, 50, 100 and 200 ng/μL). The ECO stock solution (25 mM in ethanol) and plasmid DNA stock solution (0.5 μg/μL) at predetermined amounts based on the N/P ratios were mixed and vortexed for 30 min at room temperature. Condensation of pDNA with ECO into nanoparticles was verified by agarose gel electrophoresis prepared in a 0.7% agarose gel run at 120 V for 25 min. Size and zeta potential of the nanoparticles were determined using dynamic light scattering (DLS) with an Anton Paar Litesizer 500 (Anton Paar USA, Ashland, VA). An example was given below for the characterization for nanoparticles formulated at N/P ratio of 8 with different pGRK1-ABCA4-S/MAR concentrations (FIG. 4).

The results of ECO/pGRK-ABCA4-S/MAR nanoparticle formulations were shown in FIG. 4. ECO and pGRK1-ABCA4-S/MAR could form stable nanoparticle with a size of 200 nm and a positive zeta potential of 40 mV, FIGS. 4A, B. The nanoparticles formulated also demonstrated neutral pH. Plasmid encapsulation was also confirmed by agarose gel electrophoresis FIG. 4C. ECO could efficiently encapsulate pGRK1-ABCA4-S/MAR at high concentration (200 ng/μL).

### ECO/pGRK1-ABCA4-S/MAR Nanoparticles Induced Photoreceptor Cell Specific ABCA4 Expression in Mice

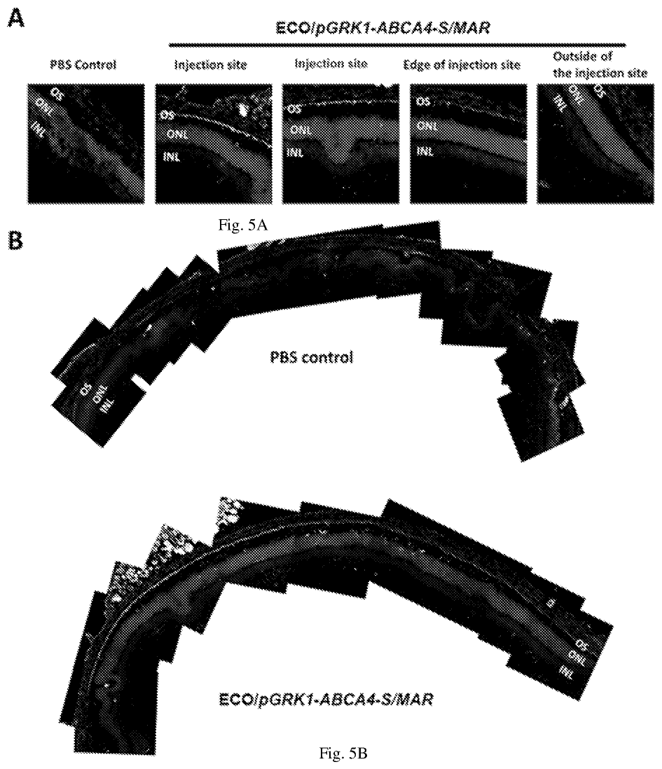

ABCA4 expression of ECO/pGRK1-ABCA4-S/MAR nanoparticle was evaluated in Abca4^{-/-}mice. ECO/pGRK1-ABCA4-S/MAR nanoparticles were formulated at N/P=8 at pDNA concentration of 200 ng/μL. The nanoparticles (1 μL) was injected into the subretinal space of the right eye, and 1 μL of PBS was injected to the left eye as the control. Mice were sacrificed after 1 week and immunohistochemistry were performed. The results were demonstrated in FIG. 5.

Immunohistochemistry demonstrated clear ABCA4 expression at or near injection site, FIG. 5A. ABCA4 expression was mainly observed in the outer segments (OS), where the photoreceptor cells are. In the whole retina, a fading of ABCA4 expression was observed from the injection site to the edge of retina, FIG. 5B. However, there is no ABCA4 expression observed for PBS injected control eye.

ABCA4 expression was also verified using qRT-PCR. Eye tissues were homogenized manually using a homogenizer in lysis buffer. RNA extraction was performed using a QIAGEN RNeasy kit according to the manufacturer's instructions. mRNA transcripts were converted to cDNA using the miScriptII reverse transcriptase kit (QIAGEN, Germantown, MD). qRT-PCR was performed with SYBR Green Master mix (AB Biosciences, Allston, MA) in an Eppendorf Mastercycler machine. Fold changes were normalized to 18S, with PBS injected eyes as controls.

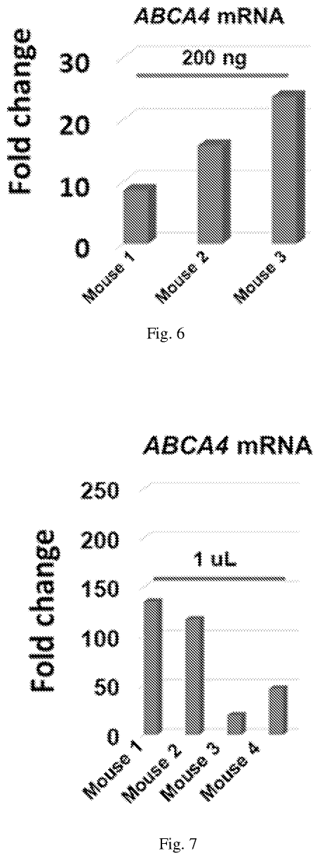

ABCA4 mRNA expression was seen in almost all the treated eyes with 200 ng nanoparticles (FIG. 6).

ECO/pGRK1-ABCA4-S/MAR Nanoparticles Formulated with Excipient (10% Sucrose) Induced ABCA4 Expression in Mice

The ECO/pGRK1-ABCA4-S/MAR nanoparticles were prepared as previously described. Sucrose (10%) was added to the nanoparticle solution and further mixed for 30 min. ECO/pGRK1-ABCA4-S/MAR nanoparticles were formulated at N/P=8 at pDNA concentration of 200 ng/μL. The nanoparticle solution (1 μL) was injected into the subretinal space of the right eye, and 1 μL of PBS was injected to the left eye as the control. Mice were sacrificed after 1 week qRT-PCR was performed.

ABCA4 mRNA expression was observed for all the treated eyes (FIG. 7). Significantly higher ABCA4 mRNA expression was observed for the treated mice than the PBS treated controls.

### Preparation of PEGylated PEG-ECO/pGRK1-ABCA4-S/MAR Nanoparticles

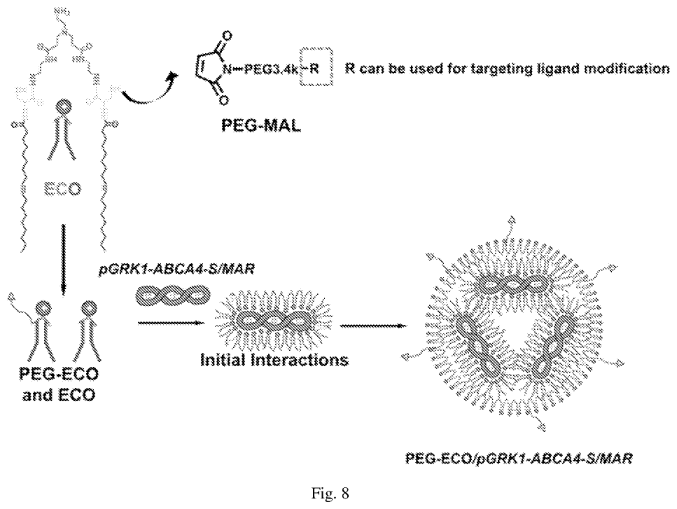

PEG-ECO/pGRK1-ABCA4-S/MAR nanoparticles were formulated as described in FIG. 8. ECO stock solution (25 mM in ethanol) and PEG-MAL targeting ligand (0.625 mM in water) (2.5 mol % of ECO) was first mixed and reacted in aqueous solution for 30 min. Plasmid DNA stock solution (0.5 μg/μL) at predetermined amounts with the N/P ratios (amine to phosphate ratio) of 6, 8 or 10 were added to ECO and PEG-MAL mixture, mixed and shaken for 30 min at room temperature to give PEG-ECO/pGRK1-ABCA4-S/MAR nanoparticles.

### Characterization of PEGylated PEG-ECO/pGRK1-ABCA4-S/MAR Nanoparticles: Size, Zeta Potential, Encapsulation and Stability in Excipient (10% Sucrose) Under Different Temperatures (−20° C. or 4° C.)

PEG-ECO/pGRK1-ABCA4-S/MAR nanoparticles were formulated as previously described. ECO stock solution (25 mM in ethanol) and PEG-MAL targeting ligand (0.625 mM in water) (2.5 mol % of ECO) was first mixed and reacted in aqueous solution for 30 min. Plasmid DNA stock solution (0.5 μg/μL) at predetermined amounts with the N/P ratios (amine to phosphate ratio) of 6, 8 or 10 were added to ECO and PEG-MAL mixture, mixed and shaken for 30 min at room temperature to give PEG-ECO/pGRK1-ABCA4-S/MAR nanoparticles. Sucrose (10%) was added to the PEG-ECO/pGRK1-ABCA4-S/MAR nanoparticle solution and shaken for additional 30 min to give ECO/pGRK1-ABCA4-S/MAR (10%) nanoparticles. ECO/pGRK1-ABCA4-S/MAR (10% sucrose) nanoparticles were prepared as described in previous section.

The sizes and zeta potentials were evaluated as previously described. The stability of the nanoparticles was evaluated under different storage conditions. All the nanoparticle formulations were stored either at −20° C. or 4° C. Size and zeta potential of the nanoparticles were determined using dynamic light scattering (DLS) with an Anton Paar Litesizer 500 (Anton Paar USA, Ashland, VA). The pDNA encapsulation was evaluated using agarose gel electrophoresis.

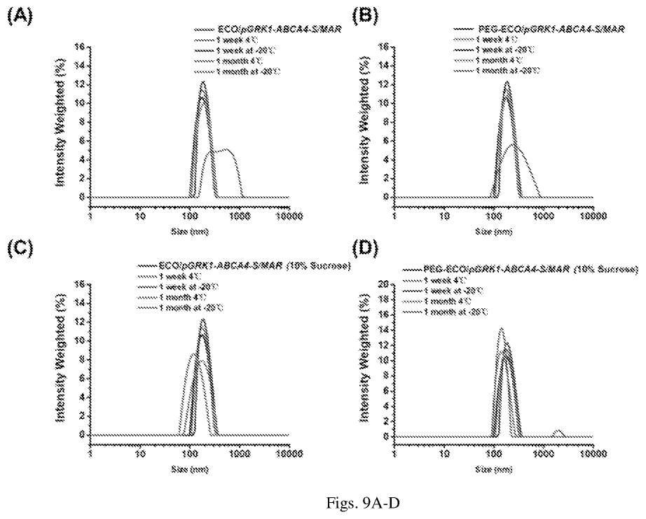

DLS of size distributions of ECO/pGRK1-ABCA4-S/MAR, PEG-ECO/pGRK1-ABCA4-S/MAR, PEG-ECO/pGRK1-ABCA4-S/MAR (10% sucrose) and PEG-ECO/pGRK1-ABCA4-S/MAR (10% sucrose) nanoparticles were summarized in FIG. 9. ECO/pGRK1-ABCA4-S/MAR nanoparticles demonstrated aggregations formation starting after a month under −20° C., when a wider size distribution was shown in FIG. 9A. There was no significant change observed for PEG-ECO/pGRK1-ABCA4-S/MAR, PEG-ECO/pGRK1-ABCA4-S/MAR (10% sucrose) and PEG-ECO/pGRK1-ABCA4-S/MAR (10% sucrose) nanoparticles FIGS. 9B, C and D. The sizes and zeta potentials were also summarized in Table 1. ECO/pGRK1-ABCA4-S/MAR nanoparticle formulations in 10% sucrose and PEGylated PEG-ECO/pGRK1-ABCA4-S/MAR nanoparticle and its formulation in 10% sucrose demonstrated consistent sizes and zeta potentials.

TABLE 1
Sizes and Zeta potentials of ECO/pGRK1-ABCA4-S/MAR and PEG-
ECO/pGRK1-ABCA4-S/MAR nanoparticles with or without 10% sucrose
under 4 or −20° C. at day 0, day 7 and 1 month.

| | | 7 day | 7 | 1 month | 1 month |
|---|---|---|---|---|---|
| Formulations | 0 day | 4° C. | day −20° C. | day 4° C. | day −20° C. |
| | Particle Size (nm) | | | | |
| ECO/pGRK1-ABCA4-S/Mar | 186.46 | 199.43 | 200.5 | 198.8 | 427 |
| PEG-ECO/pGRK1-ABCA4-S/MAR | 189.74 | 189.12 | 210.5 | 200 | 211 |
| ECO/pGRK1-ABCA4-S/MAR (10% Sucrose) | 151.15 | 173.71 | 186.6 | 125.79 | 173.92 |
| PEG-ECO/pGRK1-ABCA4-S/MAR (10% Sucrose) | 174.06 | 182.53 | 174.67 | 175.28 | 171.11 |
| | Zeta Potential (mV) | | | | |
| ECO/pGRK1-ABCA4-S/Mar | 21.8 | 26.4 | 40.3 | 30.4 | 44.6 |
| PEG-ECO/pGRK1-ABCA4-S/MAR | 13.8 | 15.1 | 22.5 | 30.4 | 28.6 |
| ECO/pGRK1-ABCA4-S/MAR (10% Sucrose) | 21.9 | 25.5 | 49.8 | 39.8 | 45 |
| PEG-ECO/pGRK1-ABCA4-S/MAR (10% Sucrose) | 15.7 | 23 | 17.7 | 27.7 | 18.7 |

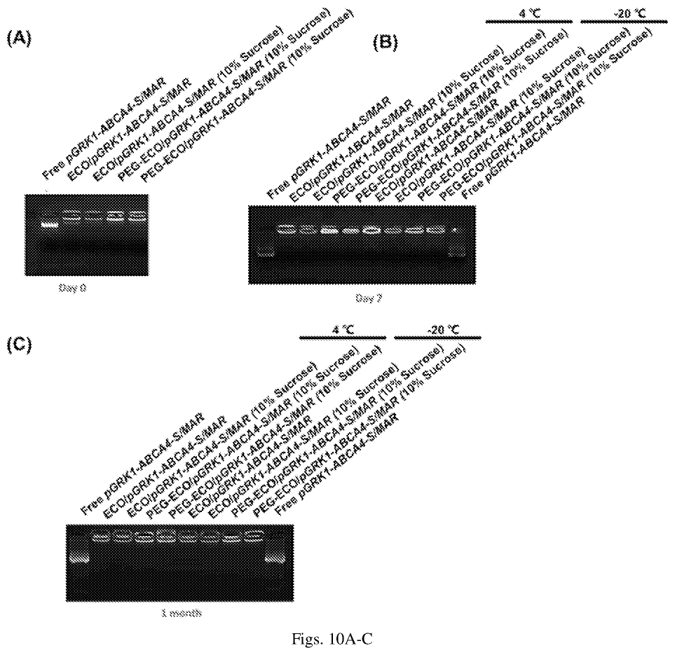

Agarose gel electrophoresis was used to evaluate the encapsulation and stability of ECO/pGRK1-ABCA4-S/MAR, PEG-ECO/pGRK1-ABCA4-S/MAR, PEG-ECO/pGRK1-ABCA4-S/MAR (10% sucrose) and PEG-ECO/pGRK1-ABCA4-S/MAR (10% sucrose) nanoparticles. Results were demonstrated in FIG. 10. All the nanoparticles demonstrated efficient pDNA encapsulation and excellent stability for all the time points tested both under 4° C. and −20° C.

PEGylated PEG-ECO/pGRK1-ABCA4-S/MAR Nanoparticles Induced High ABCA4 Expression in Mice Compared with ECO/pGRK1-ABCA4-S/MAR and ECO/pGRK1-ABCA4-S/MAR (5% Sucrose) Nanoparticles

ABCA4 expressions of different formulated nanoparticles were evaluated in Abca4^{-/-}mice. ECO/pGRK1-ABCA4-S/MAR, PEGylated (2.5% PEG) PEG-ECO/pGRK1-ABCA4-S/MAR and ECO/pGRK1-ABCA4-S/MAR (5% sucrose) nanoparticles were formulated at N/P=8 at pDNA concentration of 200 ng/μL as previous described. 5 mice were injected one eye with 0.5 μL of ECO/pGRK1-ABCA4-S/MAR nanoparticles, and the contralateral eye with 0.5 μL ECO/pGRK1-ABCA4-S/MAR (5% sucrose). 5 mice were injected one eye with 0.5 μL ECO/pGRK1-ABCA4-S/MAR nanoparticles (5% sucrose), and the contralateral eye with 0.5 μL PEGylated (2.5% PEG) PEG-ECO/pGRK1-ABCA4-S/MAR nanoparticles. Mice were sacrificed 1 week after injections. qRT-PCR was used for analysis of ABCA4 mRNA expression.

ECO/pGRK1-ABCA4-S/MAR (5% sucrose) nanoparticles demonstrated enhanced ABCA4 mRNA expression compared with ECO/pGRK1-ABCA4-S/MAR nanoparticles. PEGylated PEG-ECO/pGRK1-ABCA4-S/MAR nanoparticles induced even more ABCA4 mRNA expression than ECO/pGRK1-ABCA4-S/MAR (5% sucrose) nanoparticles (FIG. 11). PEGylated (2.5%) PEG-ECO/pGRK1-ABCA4-S/MAR nanoparticles were selected as the lead formulation for expression and treatment studies.

### Different Injection Volumes of PEG-ECO/pGRK1-ABCA4-S/MAR Nanoparticles Induced Different ABCA4 Expression in Mice

PEGylated (2.5% PEG) PEG-ECO/pGRK1-ABCA4-S/MAR nanoparticles was tested in abca4^{-/-}mice. PEG-ECO/pGRK1-ABCA4-S/MAR nanoparticles were formulated at N/P=8 at pDNA concentration of either 100 or 200 ng/μL. 5 mice were injected one eye with 1 μL of 100 ng/μL nanoparticles and the contralateral eye with 0.5 μL of 200 ng/μL nanoparticles. Mice were injected at 6 weeks old and were sacrificed 1 week after injections. qRT-PCR was used for analysis of ABCA4 mRNA expression.

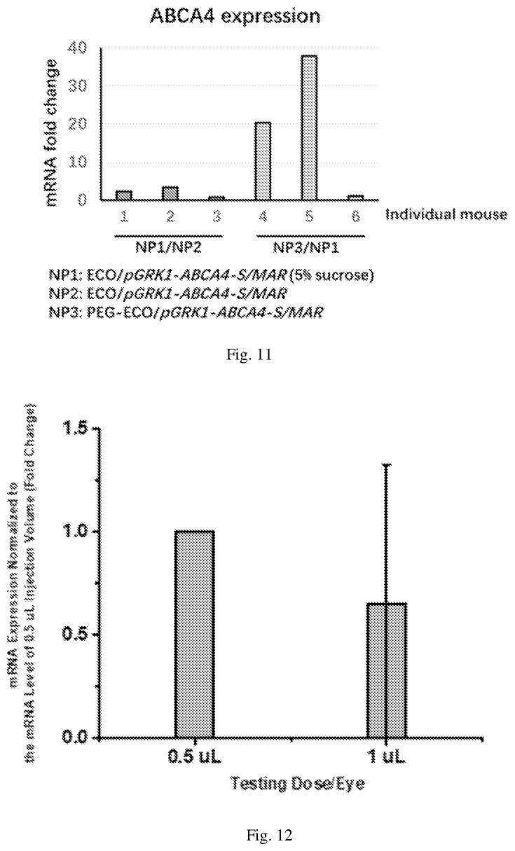

ABCA4 mRNA expression was observed for all the treated eyes (FIG. 12). Injection volume played an important role in the efficiency of ABCA4 expression. It appeared that lower injection volume introduced more ABCA4 mRNA expression. Injection volume of 1 μL demonstrated only 65% expression level compare to 0.5 μL injection volume.

### Dosing Effects of PEGylated PEG-ECO/pGRK1-ABCA4-S/MAR on ABCA4 Expression in Mice

To evaluate the dosage effect, PEGylated (2.5% PEG) PEG-ECO/pGRK1-ABCA4-S/MAR nanoparticles was tested in abca4^{-/-}mice. PEG-ECO/pGRK1-ABCA4-S/MAR nanoparticles were formulated at N/P=8 at pDNA concentration of either 50, 100 or 200 ng/μL. 5 mice were injected one eye with 0.5 μL of 50 ng/μL nanoparticles and the contralateral eye with 0.5 μL of 100 ng/μL nanoparticles. 5 mice were injected one eye with 0.5 μL of 100 ng/μL nanoparticles and the contralateral eye with 0.5 μL of 200 ng/μL nanoparticles. Mice were injected at 5 weeks old and were sacrificed 1 week after injections. qRT-PCR was used for analysis of ABCA4 mRNA expression.

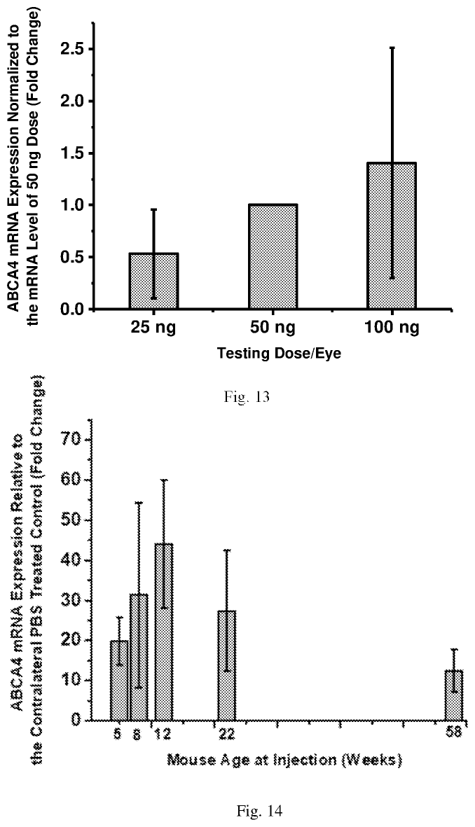

ABCA4 mRNA expression was observed for all the treated eyes (FIG. 13). An increase was observed for ABCA4 mRNA expression with the doses at the same injection volume.

### PEGylated PEG-ECO/pGRK1-ABCA4-S/MAR Induced Different ABCA4 Expression in Mice of Different Ages

PEGylated (2.5%) PEG-ECO/pGRK1-ABCA4-S/MAR nanoparticles was tested in abca4^{-/-}mice of different ages. PEGylated PEG-ECO/pGRK1-ABCA4-S/MAR nanoparticles were formulated at N/P=8 at pDNA concentration of 200 ng/μL as previously described. Mice were injected one eye with 0.5 μL of 200 ng/μL nanoparticles and the contralateral eye with 0.5 μL of PBS. Mice were injected at 5, 8, 12, 22 and 58 weeks old, and were sacrificed 1 week after injections. qRT-PCR was used for analysis of ABCA4 mRNA expression.

ABCA4 mRNA expression is low when the mice were newly born or at a young age. ABCA4 mRNA expression increased when mice were at juvenile ages or when they got slightly older. ABCA4 mRNA expression decreased when mice were at an older age (FIG. 14).

PEGylated (2.5% PEG) PEG-ECO/pGRK1-ABCA4-S/MAR Nanoparticle can Induce Comparable Gene Expression after Storage Under −20° C. for Up to 1 Month

To study the efficacy of PEGylated (2.5% PEG) PEG-ECO/pGRK1-ABCA4-S/MAR nanoparticles stored for different periods of time under −20° C., PEGylated (2.5% PEG) PEG-ECO/pGRK1-ABCA4-S/MAR nanoparticles was tested in abca4^{-/-}mice. PEG-ECO/pGRK1-ABCA4-S/MAR nanoparticles were formulated at N/P=8 at pDNA concentration of 200 ng/μL. The nanoparticles were kept under −20° C. 1 week, 2 weeks and 1 month. The nanoparticles were then injected to abca4^{-/-}mice with the injection volume of 0.5 μL (100 ng dose) through subretinal injection. Mice were injected at 3 months old and were sacrificed 2 weeks and 1 month after injections. qRT-PCR was used for analysis of ABCA4 mRNA expression.

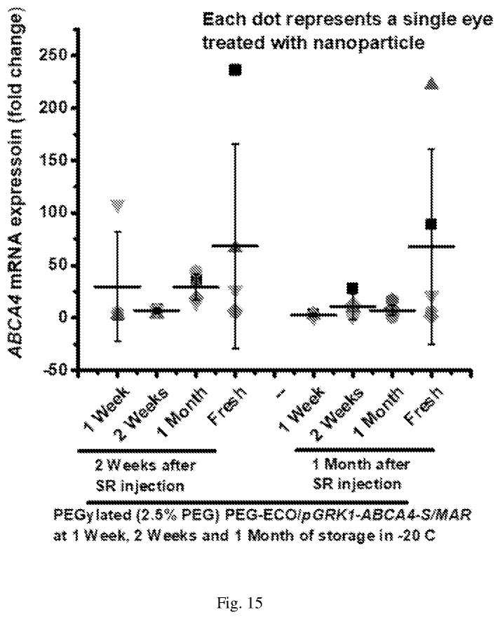

ABCA4 mRNA expression was observed for almost all the eyes treated by subretinal injections of PEGylated (2.5% PEG) PEG-ECO/pGRK1-ABCA4-S/MAR nanoparticles (FIG. 15). ABCA4 mRNA expression didn't demonstrate significant difference for nanoparticles stored for 1 week, 2 weeks and 1 month on average. However, compared with fresh nanoparticles, a reduction in ABCA4 mRNA expression was observed on average for all the nanoparticles kept under −20° C. However, statistically, there is no significant difference.

### PEGylated (2.5% PEG) PEG-ECO/pGRK1-ABCA4-S/MAR Nanoparticle can Induce Gene Expression Through Both Subretinal and Intravitreal Injections

To demonstrate the efficacy of PEGylated (2.5% PEG) PEG-ECO/pGRK1-ABCA4-S/MAR nanoparticles through different injection routes, PEGylated (2.5% PEG) PEG-ECO/pGRK1-ABCA4-S/MAR nanoparticles was tested in abca4^{-/-}mice. PEG-ECO/pGRK1-ABCA4-S/MAR nanoparticles were formulated at N/P=8 at pDNA concentration of 200 ng/μL. 10 mice were injected one eye with 0.5 μL of 200 ng/μL nanoparticles (previously identified dose and injection volume) through subretinal injection and the contralateral eye with 0.5 μL of 200 ng/μL nanoparticles through intravitreal injection. Mice were injected at 2 months old and were sacrificed 2 weeks and 1 month after injections. qRT-PCR was used for analysis of ABCA4 mRNA expression.

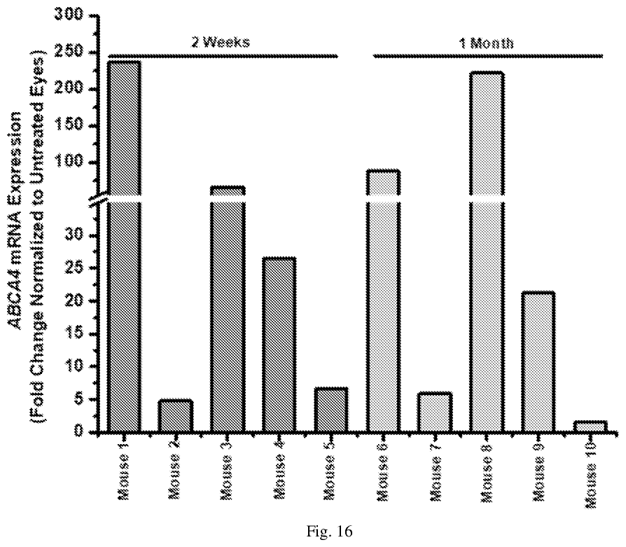

The results of ABCA4 mRNA expression through subretinal injection were summarized in FIG. 16. 9 out of 10 mice demonstrated significantly more ABCA4 mRNA expression than non-treated control mice. The A2E analysis is currently on going using previously selected treatment dose of 100 ng/eye and injection volume of 0.5 μL due to the time frame of the evaluation (6 months).

To demonstrated the efficacy of PEGylated (2.5% PEG) PEG-ECO/pGRK1-ABCA4-S/MAR nanoparticles through different injection routes, PEGylated (2.5% PEG) PEG-ECO/pGRK1-ABCA4-S/MAR nanoparticles was tested in abca4^{-/-}mice. PEG-ECO/pGRK1-ABCA4-S/MAR nanoparticles were formulated at N/P=8 at pDNA concentration of 200 ng/μL. 10 mice were injected one eye with 0.5 μL of 200 ng/μL nanoparticles (previously identified dose and injection volume) through subretinal injection and the contralateral eye with 0.5 μL of 200 ng/μL nanoparticles through intravitreal injection. Mice were injected at 2 months old and were sacrificed 2 weeks and 1 month after injections. qRT-PCR was used for analysis of ABCA4 mRNA expression.

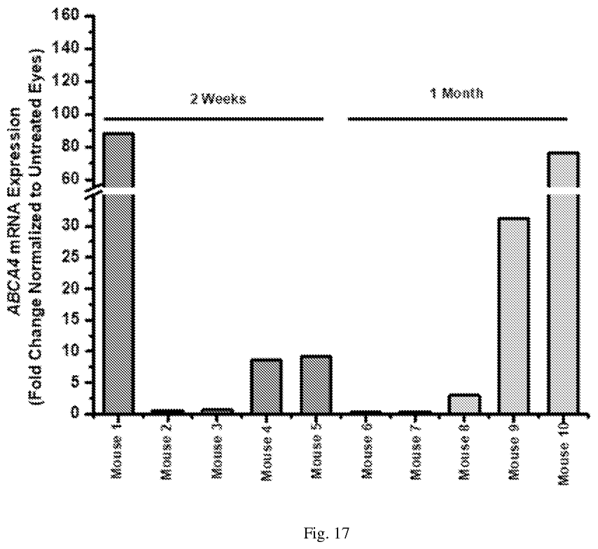

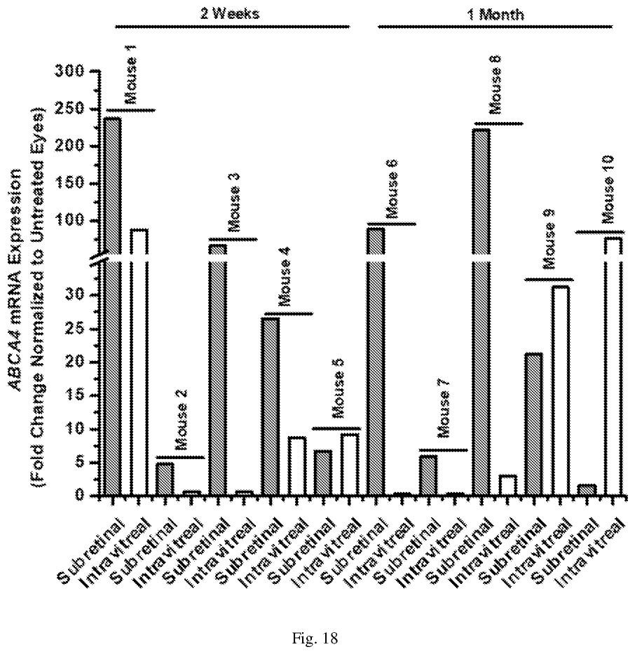

The results of ABCA4 mRNA expression through intravitreal injection were summarized in FIG. 17. 6 out of 10 mice demonstrated significantly more ABCA4 mRNA expression than non-treated control mice. The comparison between subretinal and intravitreal injection was summarized in FIG. 18. Overall, subretinal injection demonstrated better efficacy than intravitreal injection. Surprisingly, intravitreal administration of PEG-ECO/pGRK1-ABCA4-S/MAR nanoparticles could also induce ABCA4 expression in the outer retina, which raised the potential application of ECO based nanoparticle formulation for intravitreal administration. However, due to the limited efficacy compared with subretinal injection route, the A2E analysis was only conducted using subretinal injection route for PEGylated ECO nanoparticles at previously determined dose 100 ng/eye and 0.5 μL injection volume.

ECO Based Gene Therapy Using PEG-ECO/pGRK1-ABCA4-S/MAR Nanoparticles can Prevent A2E Accumulation in Abca4^{-/-}Mice with a Single Treatment for 8 Months and 1 Year

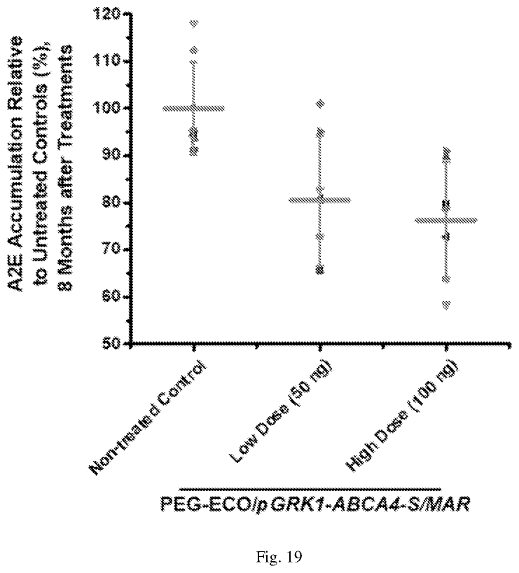

To demonstrate the efficacy of ECO based gene therapy after a single treatment, PEGylated (2.5% PEG) PEG-ECO/pGRK1-ABCA4-S/MAR nanoparticles on A2E was tested in abca4^{-/-}mice (1-2.5 months old). PEG-ECO/pGRK1-ABCA4-S/MAR nanoparticles were formulated at N/P=8 at pDNA concentration of 100 ng or 200 ng/μL. The nanoparticles were then injected to abca4^{-/-}mice with the injection volume of 0.5 μL (100 ng and 50 ng dose) through subretinal injection. Mice were sacrificed 8 months or 1 year after injections. HPLC was used to analyze the A2E amount in each eye ball. qRT-PCR was used to evaluate the ABCA4 mRNA expression and immunohistochemistry was used to demonstrate ABCA4 protein expression. Results were shown in FIGS. 19, 20, 21, 22 and 23.

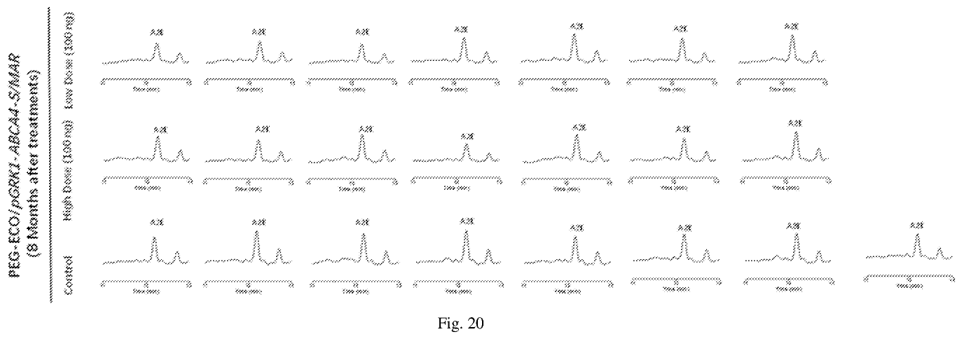

A reduction of about 25% in A2E accumulation was observed for nanoparticle treated abca4^{-/-}mice with high dose (100 ng/eye) compared with the untreated controls. A reduction of 20% in A2E accumulation was observed for nanoparticle treated mice with low dose (50 ng/eye). On average, gene therapy with a higher dose can result in better prevention but not significantly different from the low dose selected in this experiment (FIG. 19). The chromatograms from the HPLC analysis also demonstrated smaller peaks from the treated groups than the control groups (FIG. 20).

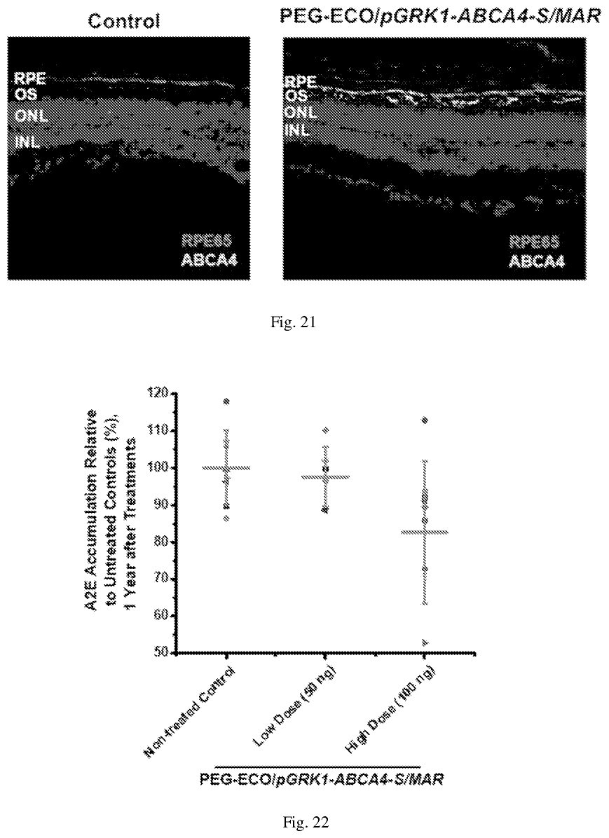

ABCA4 protein expression was also observed 8 months after a single treatment of EG-ECO/pGRK1-ABCA4-S/MAR nanoparticles in abca4^{-/-}mice. With the help of immunohistochemistry, ABCA4 protein (labeled in green) expression could be observed predominantly in the photoreceptor outer segment (OS) due to the incorporation of the cell specific promoter GRK1 (FIG. 21).

Similar reduction of 20% in A2E accumulation was observed for nanoparticle treated abca4^{-/-}mice with high dose (100 ng/eye). Slight reduction of A2E accumulation was observed for nanoparticle treated mice with low dose (50 ng/eye), but not significantly different from the control group. On average, gene therapy with a higher dose can result in better prevention (FIG. 22).

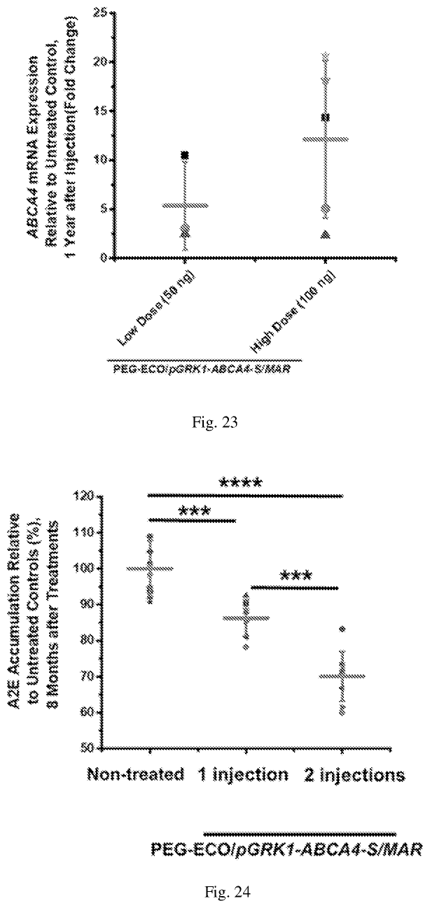

ABCA4 mRNA expression was also observed 1 year after a single treatment of EG-ECO/pGRK1-ABCA4-S/MAR nanoparticles in abca4^{-/-}mice, with higher mRNA expression for the mice treated with high dose than the mice treated with low dose on average but not significantly different (FIG. 23).

ECO Based Gene Therapy Using PEG-ECO/pGRK1-ABCA4-S/MAR Nanoparticles can Prevent A2E Accumulation in Abca4^{-/-}Mice with Multi-Treatments 8 Months after the First Treatment

To demonstrate the efficacy of ECO based gene therapy after a single treatment, PEGylated (2.5% PEG) PEG-ECO/pGRK1-ABCA4-S/MAR nanoparticles on A2E was tested in abca4--mice (1-2.5 months old). PEG-ECO/pGRK1-ABCA4-S/MAR nanoparticles were formulated at N/P=8 at pDNA concentration of 100 ng or 200 ng/μL. The nanoparticles were then injected to abca4^{-/-}mice with the injection volume of 0.5 μL (100 ng) through subretinal injection. For single injection, the mice were only injected once. For multi-injections, the mice were injected every 3 months. Mice were sacrificed 8 months after the first treatment. HPLC was used to analyze the A2E amount in each eye ball. qRT-PCR was used to evaluate the ABCA4 mRNA expression. Results were shown in FIGS. 19, 20, 21 and 22.

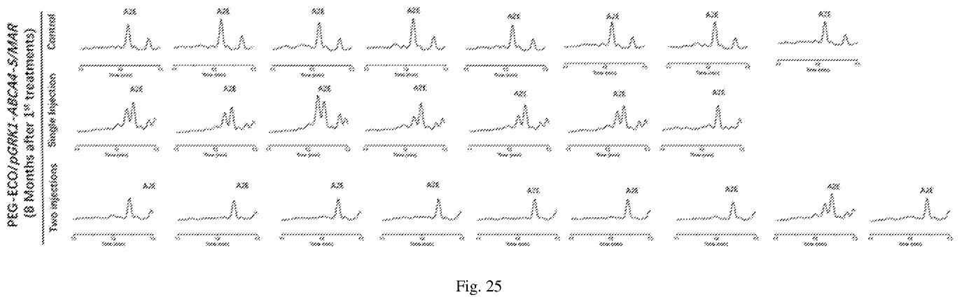

A reduction of about 16% in A2E accumulation was observed for abca4^{-/-}mice treated with single injection of nanoparticles. About 30% reduction was observed for mice injected with 2 doses of nanoparticles. Both were significantly different from the control groups. The preventive effect is significantly better for the multi-treatment group than the single injection (FIG. 24). The chromatograms from the HPLC analysis also demonstrated smaller peaks from the treated groups than the control groups, especially for the multi-treatment group (FIG. 25).

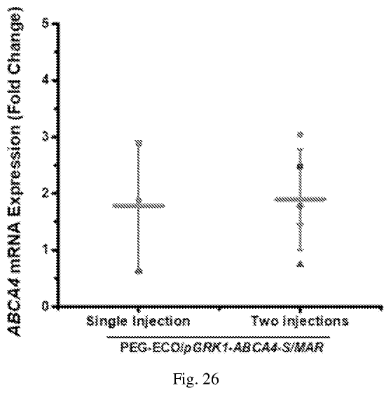

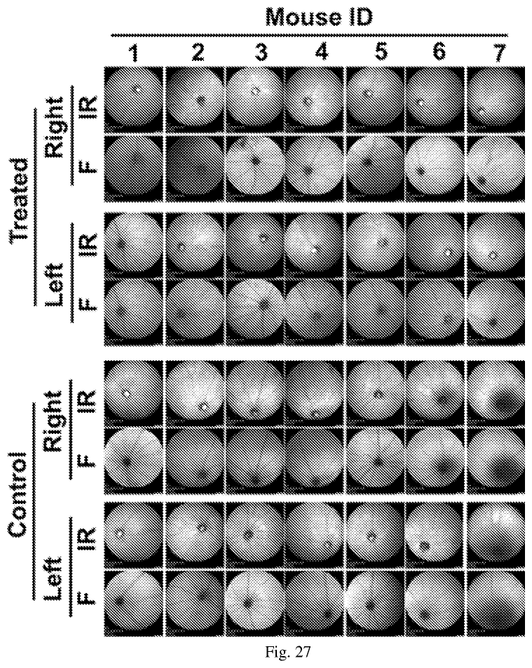

ABCA4 mRNA expression was also observed for both single and multi-treatment groups in abca4^{-/-}mice. No significant difference of expression was observed for the two groups on the RNA level (FIG. 26). Further histological studies will be performed to evaluate ABCA4 expression on protein level.

ECO Based Gene Therapy Using PEG-ECO/pGRK1-ABCA4-S/MAR Nanoparticles Demonstrated Excellent Safety Profiles in Abca4^{-/-}Mice after Single or Multi-Treatments

To demonstrate the safety of ECO based gene therapy after a single treatment, PEGylated (2.5% PEG) PEG-ECO/pGRK1-ABCA4-S/MAR nanoparticles on A2E was tested in abca4--mice (1-2.5 months old). PEG-ECO/pGRK1-ABCA4-S/MAR nanoparticles were formulated at N/P=8 at pDNA concentration of 200 ng/μL. The nanoparticles were then injected to abca4^{-/-}mice with the injection volume of 0.5 μL (100 ng) through subretinal injection. The eye condition was evaluated using scanning laser ophthalmoscopy (SLO) 7 months after a single dose injection or 8 months after 2 injections. No treatment associated adverse effect was observed for the gene therapy. Some retinal degenerative events were observed for the control groups, demonstrated by the dark area near the optic nerve (FIG. 27).

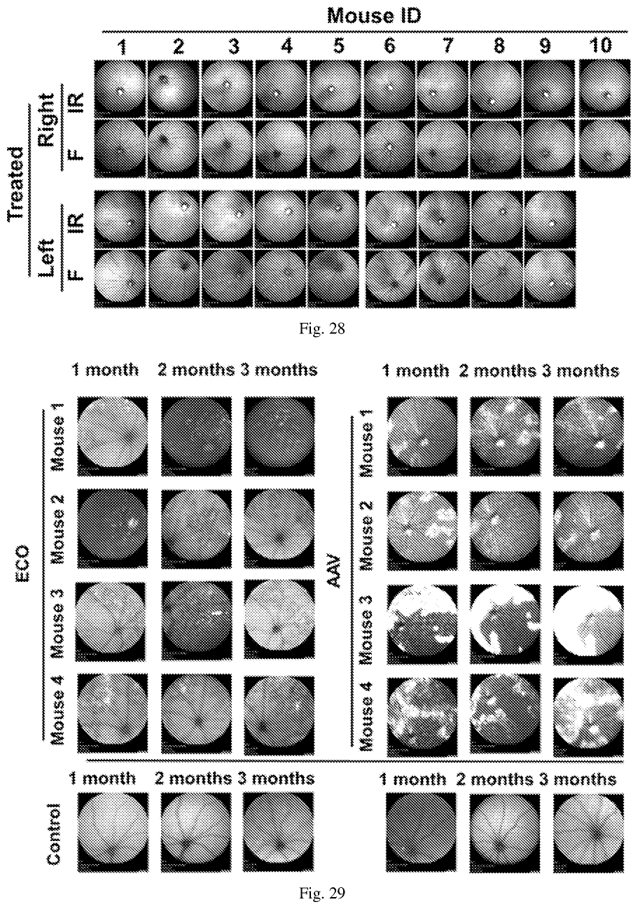

Similarly, no significant treatment associated adverse effect was observed for the gene therapy. Slight dark areas were observed in the SLO images and could recover. (FIG. 28).

ECO Based Nanoparticle Platform Demonstrated Better Safety Profile than Type 2 Adeno-Associated Virus (AAV2) Based System

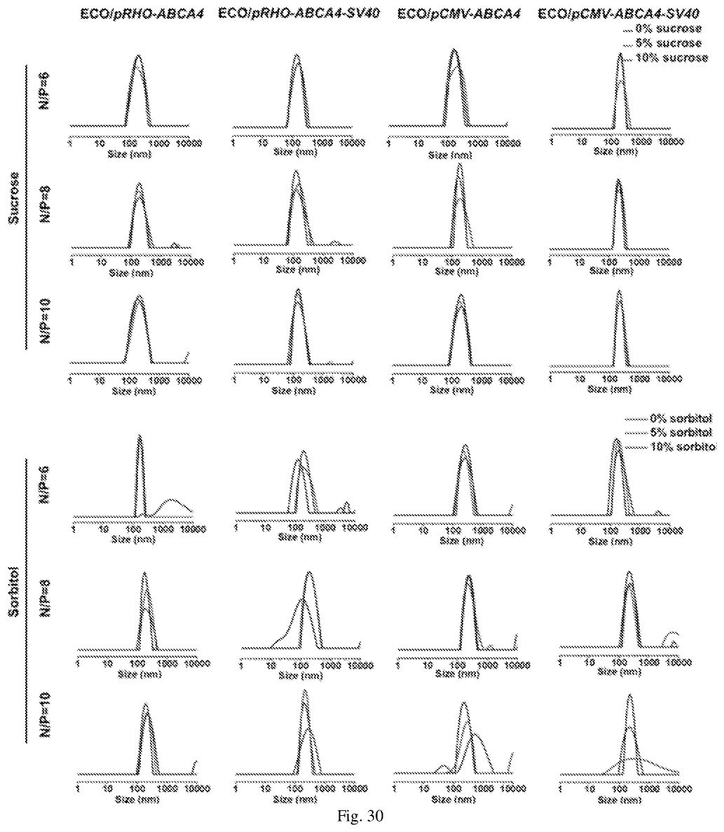

To evaluate the safety of ECO based nanoparticle system in comparison with AAV2 based system, formulated PEGylated ECO nanoparticle in 5% sucrose was selected using reporter GFP plasmid (pCMV-GFP) with AAV2-CMV-GFP as a control. ACU-PEG-HZ-ECO/pCMV-GFP nanoparticles were formulated at N/P=8 at pDNA concentration of 50 ng/μL with 5% sucrose. The nanoparticles (0.5 μL) was injected into the subretinal space of one eye of a BALB/c mouse. The contralateral eye was injected with AAV2-CMV-GFP of the same dose. Untreated mice were used as controls. Scanning laser ophthalmoscope (SLO) was used to evaluate the eye condition and GFP expression at 1, 2 and 3 months (FIG. 29). GFP expression was observed for both formulated ECO nanoparticles and AAV2 systems at all time points.

AAV2 seemed to induce more lesions and potential inflammations than ECO nanoparticles demonstrated by the saturated white color regions. ECO based nanoparticle platform didn't demonstrate severe adverse events and the eye condition could recover from the treatments.

ECO can Formulate Stable Nanoparticles with pDNA of Different Sizes in 5% Sucrose as Excipient Compared to Sorbitol

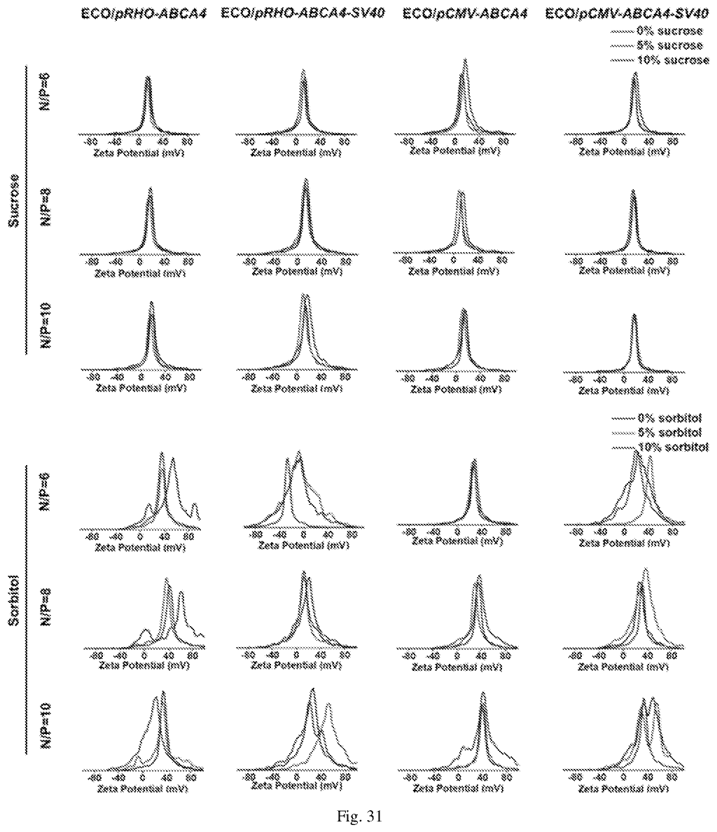

ECO/pDNA nanoparticles of pRHO-ABCA4-SV40 and pCMV-ABCA4-SV40 were first prepared by self-assembly of ECO with the plasmids at amine/phosphate (N/P) ratios of 6, 8 and 10. The plasmids pRHO-ABCA4 and pCMV-ABCA4 were used as controls. Sucrose or sorbitol were then added as an excipient with a concentration of 5% or 10% to stabilize the nanoparticles. The nanoparticles were characterized by dynamic light scattering (DLS) for their size distributions (FIG. 30) and zeta potential distributions (FIG. 31) under different excipient conditions. As shown in FIG. 30, ECO and pABCA4s formulated stable nanoparticles at all the N/P ratios in the presence of sucrose at both 5% and 10% contents. The sizes were between 220 nm and 250 nm and the distributions were seen as single peaks with negligible aggregation peaks and were not affected by the excipient. However, sorbitol addition affected some of the ECO/pABCA4 formulations. For example, ECO/pRHO-ABCA4 at N/P ratio of 6, ECO/pRHO-ABCA4-SV40 at N/P ratio of 8, ECO/pCMV-ABCA4 at N/P ratio of 10, and ECO/pCMV-ABCA4-SV40 at N/P ratio of 10 showed broadened size distributions after sorbitol addition. Zeta potential distributions demonstrated no noticeable change after sucrose additions for the ECO/pABCA4 nanoparticle formulations (FIG. 31). ECO/pABCA4 nanoparticles demonstrated uniformed distributions around +20 mV across all the N/P ratios under sucrose conditions. However, sorbitol addition to ECO/pABCA4 nanoparticle formulations resulted in some noticeable changes in zeta potential distributions.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A plasmid vector comprising one or more heterologous nucleic acids encoding a functional therapeutic protein configured to treat a retinal or ocular disorder, the plasmid vector comprising a human GRK1 promoter and human S/MAR enhancer to express the heterologous gene in both rod and cone photoreceptors.

2: The plasmid vector of claim 1, wherein the GRK1 promoter is upstream of the heterologous gene and the S/MAR enhancer is downstream of the heterologous gene.

3: The plasmid vector of claim 1, further including a human beta-globin polyadenosine (polyA) signal sequence.

4: The plasmid vector of claim 1, wherein the beta-globin polyA signal sequence is downstream of the heterologous gene and upstream of the S/MAR enhancer.

5: The plasmid vector of claim 1, wherein the retinal disorder is an inheritable retinal disorder caused by a mutation of a gene encoding an ocular protein.

6: The plasmid vector of claim 1, wherein the inheritable retinal disorder is selected from Stargardt Disease, Leber's congenital amaurosis (LCA), pseudoxanthoma elasticum, rod cone dystrophy, exudative vitreoretinopathy, Joubert Syndrome, CSNB-1 C, retinitis pigmentosa, stickler syndrome, microcephaly, choriorretinopathy, CSNB 2, Usher syndrome, Wagner syndrome, or age-related macular degeneration.

7: The plasmid vector of claim 1, wherein the heterologous gene is selected from Retinal pigment epithelium-specific 65 kDa protein (RPE 65), vascular endothelial growth factor (VEGF) inhibitor or soluble VEGF receptor 1 (sFif1), (Rab escort protein-1) REP1, L-opsin, rhodopsin (Rho), phosphodiesterase 6(3 (PDE613), ATP-binding cassette, sub-family A, member 4 (ABCA4), lecithin retinol acyltransferase (LRAT), Retinal degeneration, slow/Peripherin (RDS/Peripherin), Tyrosine-protein kinase Mer (MERTK), Inosine-5 prime-monophosphate dehydrogenase, type I (IMPDHI), guanylate cyclase 2D (GUCY2D), aryl-hydrocarbon interacting protein-like 1 (AIPL 1), retinitis pigmentosa GTPase regulator interacting protein 1 (RPGRIPI), Inosine-5-prime-monophosphate dehydrogenase, type I (IMPDH1 guanine nucleotide binding protein, alpha transducing activity polypeptide 2 (GNAT2), cyclic nucleotide gated channel beta 3 (CNGB3), retinoschisin 1 (Rs1), ocular albinism type 1 (OA1), oculocutaneous albinism type 1 (OCA1) tyrosinase, P21 WAF-1/Cip1, platelet-derived growth factor (PDGF), Endostatin Angiostatin, arylsulfatase B, 13-glucuronidase, usherin 2A (USH2A), CEP290, ABCC, RIMS1, LRP5, CC2D2A, TRPM1, IFT-172, COL11A1, TUBGCP6, KIAA1549, CACNA1F, MYO7A, VCAN, or HMCN1.

8: The plasmid vector of claim 1, wherein the retinal disorder is Stargardt Disease.

9: The plasmid vector of claim 8, wherein the heterologous gene is ABCA4.

10: The plasmid vector of claim 1, having the nucleic acid sequence of SEQ ID NO: 1.

11: A self-assembled nanoparticle comprising a plurality of pH sensitive multifunctional cationic lipids complexed with one or more plasmid vector(s) of claim 1.

12: The self-assembled nanoparticle of claim 11, wherein the pH sensitive multifunctional cationic lipids comprise pH sensitive multifunctional amino lipids.

13: The self-assembled nanoparticle of claim 12, having an amine to phosphate (N/P) ratio of about 4 to about 12.

14: The self-assembled nanoparticle of claim 11, wherein the pH sensitive multifunctional cationic lipids further include at least one targeting group that targets and/or binds to a retinal or visual protein.

15: The self-assembled nanoparticles of claim 14, wherein the at least one targeting group includes a retinoid or retinoid derivative that targets and/or binds to an interphotoreceptor retinoid binding protein.

16: The self-assembled nanoparticles of claim 14, wherein the at least one targeting group includes all-trans-retinylamine or (1R)-3-amino-1-[3-(cyclohexylmethoxy)phenyl]propan-1-ol.

17: The self-assembled nanoparticles of claim 14, wherein the pH sensitive multifunctional cationic lipids include a cysteine residue and the at least one targeting group is covalently attached to a thiol group of the cysteine residue by a linker.

18: The self-assembled nanoparticles of claim 17, wherein the linker comprises a polyamino acid group, a polyalkylene group, or a polyethylene glycol group.

19: The self-assembled nanoparticles of claim 18, wherein the linker comprises an acid labile bond.

20: The self-assembled nanoparticles of claim 14, further being PEGylated.

21: The self-assembled nanoparticles of claim 14, wherein the pH sensitive multifunctional cationic lipids comprise (1-aminoethyl)iminobis[N-(oleoylcysteinyl-1-amino-ethyl)propionamide) (ECO) or an analogue or derivative thereof.

22-35. (canceled)

36: A pharmaceutical composition comprising an aqueous solution of the self-assembled nanoparticles of claim 11.

37-59. (canceled)


SEQUENCE LISTING (1 sequence)

SEQ ID NO: 1 - DNA, 9993 bp, Artificial Sequence
  Synthetic Construct
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggcaagggcaccggtatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgagcggccgcacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcactagtattcgaattcagcaaggtcgccacgcattcgaattcagcaaggtcgccacgcacaagatcaatattaacaatcagtcatctctctttagcaataaaaaggtgaaaaattacattttaaaaatgacaccatagacgatgtatgaaaataatctacttggaaataaatctaggcaaagaagtgcaagactgttacccagaaaacttacaaattgtaaatgagaggttagtgaagatttaaatgaatgaagatctaaataaacttataaattgtgagagaaattaatgaatgtctaagttaatgcagaaacggagagacatactatattcatgaactaaaagacttaatattgtgaaggtatactttcttttcacataaatttgtagtcaatatgttcaccccaaaaaagctgtttgttaacttgtcaacctcatttcaaaatgtatatagaaagcccaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagtcattttcatatttttcttgaacattaagtacaagtctataattaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgataga